(19) United States
(12) Patent Application Publication
(10) Pub. No.: US 2018/0104232 A1
(43) Pub. Date: Apr. 19, 2018

(54) METHODS FOR DETERMINING PROGNOSIS FOR BREAST CANCER PATIENTS

(71) Applicant: The University of Chicago, Chicago, IL (US)

(72) Inventors: Marsha Rosner, Chicago, IL (US); Casey Frankenberger, Chicago, IL (US); Russell Bainer, Chicago, IL (US); Daniel Rabe, Chicago, IL (US)

(21) Appl. No.: 15/556,474

(22) PCT Filed: Mar. 11, 2016
(86) PCT No.: PCT/US2016/021972

§ 371 Date: Sep. 7, 2017

Related U.S. Application Data
(60) Provisional application No. 62/132,222, filed on Mar. 12, 2015.

(51) Int. Cl. A61K 31/46; A61P 35/00; A61K 31/517; A61K 45/06; A61K 39/395

(52) U.S. Cl. CPC A61K 31/46; A61P 35/00; A61K 2039/505; A61K 45/06; A61K 39/3955; A61K 31/517

(57) ABSTRACT

Described herein are methods for treating breast cancer and for providing a prognosis for metastatic-free survival of breast cancer patients. Aspects relate to treating a patient determined to be at high risk for developing or having metastatic breast cancer comprising administering adjuvant or neoadjuvant therapy to the patient determined to be at high risk for developing or having metastatic breast cancer, wherein the patient was determined to be at high risk for developing or having metastatic breast cancer by determining that the expression level of RKIP was reduced and/or the expression level of one or more of HMGA2, CCL5, TN-FR2, GRN, and CCL7 was elevated in a biological sample from the patient compared to a control non-metastatic tissue sample.

### CROSS-REFERENCE TO RELATED APPLICATIONS

This application claims the benefit of priority of U.S. Provisional Patent Application No. 62/132,222, filed Mar. 12, 2015, which is hereby incorporated by reference in its entirety.

### STATEMENT OF GOVERNMENT SUPPORT

This invention was made with government support under R01GM087630-17 awarded by the National Institutes of Health. The government has certain rights in the invention.

### BACKGROUND

### 1. Field of the Invention

Embodiments are directed generally to biology and medicine. In certain aspects methods involve treating breast cancer patients and determining the prognosis for a breast cancer patient. In additional embodiments there are therapeutic compositions and the use of such compositions for the treatment of breast cancer.

### 2. Background

There are over 1 million cases of breast cancer per year on a global basis, of which around 0.5 million are in the US, 40,000 are in the UK and nearly 2,000 in Ireland. It is the leading cause of cancer deaths among women (Keen and Davidson, 2003). Although the overall incidence of the disease is increasing within the western world, wider screening and improved treatments have led to a gradual decline in the fatality rate of about 1% per year since 1991. Inheritance of susceptibility genes, such as BRCA1 and BRCA2, account for only 5% of breast cancer cases and the factors responsible for the other 95% remain obscure (Grover and Martin, 2002).

Mere classification of breast cancers into a few subgroups characterized by low to absent gene expression of the estrogen receptor (ER) alone may not reflect the cellular and molecular heterogeneity of breast cancer, and may not allow the design of treatment strategies maximizing patient response. Once a patient is diagnosed with cancer, such as breast or ovarian cancer, or an individual wants predisposition analysis, there is a strong need for methods that allow the physician to predict the expected course of disease, including the likelihood of cancer recurrence, long-term survival of the patient, and the like, and accordingly select an appropriate treatment option that is effective.

### SUMMARY OF THE DISCLOSURE

The current disclosure fulfills the aforementioned need in the art by providing improved therapeutic methods for treating breast cancer patients and a gene signature that can be used as a prognostic predictor for metastatic-free survival.

Aspects of the disclosure relate to a method for treating a patient determined to be at high risk for developing or having metastatic breast cancer comprising: administering adjuvant or neoadjuvant therapy to the patient determined to be at high risk for developing or having metastatic breast cancer, wherein the patient was determined to be at high risk for developing or having metastatic breast cancer by determining that the expression level of RKIP was reduced and/or the expression level of one or more of HMGA2, CCL5, and TAM-metagene was elevated in a biological sample from the patient compared to a control non-metastatic tissue sample.

Raf kinase inhibitor protein (RKIP) is a kinase inhibitor protein, that regulates many signaling pathways within the cell. RKIP is also known as HCNP, HCNPpp, HEL-210, HEL-S-34, PBP, PEBP, and PEBP-1 in humans. The human protein sequence is represented by GenBank Accession No: NP_002558, and the mRNA sequence is represented by GenBank Accession No: NM_002567.2. The sequence associated with each of these GenBank Accession Nos. are incorporated by reference.

High-mobility group AT-hook 2, also known as HMGA2, is a protein that, in humans, is encoded by the HMGA2 gene. HMGA2 is also known as BABL, HMGI-C, HMGIC, LIPO, and STQTL9 in humans. The human protein sequence is represented by GenBank Accession No: NP_001287847, and the mRNA sequence is represented by GenBank Accession No: NM_001015886. The sequence associated with each of these GenBank Accession Nos. are incorporated by reference.

Chemokine (C-C motif) ligand 5 (also CCL5) is a protein which in humans is encoded by the CCL5 gene. CCL5 is also known as D17S136E, RANTES, SCYA5, SIS-delta, SISd, TCP228, and eoCP in humans. The human protein sequence is represented by GenBank Accession No: NP_001265665, and the mRNA sequence is represented by GenBank Accession No: NM_001278736. The sequence associated with each of these GenBank Accession Nos. are incorporated by reference.

TAM-metagene refers to the expression level of TNFR2, GRN, and CCL7.

Tumor necrosis factor receptor superfamily member 1B (TNFRSF1B) also known as tumor necrosis factor receptor 2 (TNFR2) and CD120b is a protein that in humans is encoded by the TNFRSF1B gene. The human protein sequence is represented by GenBank Accession No: NP_001057, and the mRNA sequence is represented by GenBank Accession No: NM_001066. The sequence associated with each of these GenBank Accession Nos. are incorporated by reference.

Granulin is a protein that in humans is encoded by the GRN gene. GRN is also known as CLN11, GEP, GP88, PCDGF, PEPI, and PGRN in humans. The human protein sequence is represented by GenBank Accession No: NP_002078, and the mRNA sequence is represented by GenBank Accession No: NM_001012479. The sequence associated with each of these GenBank Accession Nos. are incorporated by reference.

Chemokine (C-C motif) ligand 7 (CCL7) is a small cytokine known as a chemokine that was previously called monocyte-specific chemokine 3 (MCP3). CCL7 is also known as FIC, MARC, MCP-3, MCP3, NC28, SCYA6, and SCYA7 in humans. The human protein sequence is represented by GenBank Accession No: NP_006264, and the mRNA sequence is represented by GenBank Accession No: NM_006273. The sequence associated with each of these GenBank Accession Nos. are incorporated by reference.

Adjuvant therapy for breast cancer is any treatment given after primary therapy to increase the chance of long-term disease-free survival. Primary therapy is the main treatment used to reduce or eliminate the cancer. Primary therapy for breast cancer usually includes surgery—a mastectomy (removal of the breast) or a lumpectomy (surgery to remove the tumor and a small amount of normal tissue around it; a type of breast-conserving surgery). During either type of surgery, one or more nearby lymph nodes are also removed to see if cancer cells have spread to the lymphatic system. When a woman has breast-conserving surgery, primary therapy almost always includes radiation therapy.

Neoadjuvant therapy is treatment given before primary therapy. A woman may receive neoadjuvant chemotherapy for breast cancer to shrink a tumor that is inoperable in its current state, so it can be surgically removed. A woman whose tumor can be removed by mastectomy may instead receive neoadjuvant therapy to shrink the tumor enough to allow breast-conserving surgery.

In some embodiments, the adjuvant or neoadjuvant therapy comprises one or more of chemotherapy, hormonal therapy, surgical removal of the breast and/or ovaries, trastuzumab, and radiation therapy. In some embodiments, the chemotherapy comprises one or more of docetaxel, paclitaxel, cisplatin, carboplatin, vinorelbine, capecitabine, liposomal doxorubicin, gemcitabine, mitoxantrone, ixabepilone, albumin-bound paclitaxel, trastuzumab, tamoxifen, aromatase inhibitor, toremifene, magestrol acetate, fluvestran, and eribulin. In some embodiments, the aromatase inhibitor comprises one or more of letrozole, anastrozole, and exemstane. It is specifically contemplated that one or more of these may be excluded as a treatment option in certain embodiments. In some embodiments, an adjuvant therapy is administered to the patient determined to be at high risk for developing or having metastatic breast cancer. In some embodiments, the patient was determined to be at high risk for developing or having metastatic breast cancer when at least three of the following are determined: reduced RKIP expression, elevated HMGA2 expression, elevated CCL5 expression, and elevated TAM-metagene expression.

Further aspects relate to a method for treating a patient for metastatic or non-metastatic breast cancer comprising: treating the patient for metastatic breast cancer after the patient is determined to have an elevated expression level of one or more of HMGA2, CCL5, and TAM-metagene and/or a reduced expression level of RKIP in a biological sample from the patient relative to the expression level of the same genes in a control non-metastatic tissue sample; and treating the patient for non-metastatic breast cancer after the patient is determined to have a reduced or substantially the same expression level of one or more of HMGA2, CCL5, and TAM-metagene and/or an elevated or substantially the same expression level of RKIP in a biological sample from the patient relative to the expression level of the same genes in a control non-metastatic tissue sample.

The term “substantially the same” refers to a level of expression or protein activity that is not significantly different to another level of expression or protein activity.

In some embodiments, the treatment for metastatic breast cancer comprises one or more of chemotherapy, hormonal therapy, surgical removal of the breast and/or ovaries, trastuzumab, and radiation therapy. In some embodiments, the treatment for the patient with non-metastatic breast cancer excludes one or more of chemotherapy, surgical removal of the breast and/or ovaries, and radiation therapy. In some embodiments, the chemotherapy comprises one or more of docetaxel, paclitaxel, cisplatin, carboplatin, vinorelbine, capecitabine, liposomal doxorubicin, gemcitabine, mitoxantrone, ixabepilone, albumin-bound paclitaxel, trastuzumab, tamoxifen, aromatase inhibitor, toremifene, magestrol acetate, fluvestran, and eribulin. In some embodiments, the aromatase inhibitor comprises one or more of letrozole, anastrozole, and exemstane. In some embodiments, the treatment for the patient with metastatic breast cancer comprises adjuvant therapy. In some embodiments, the treatment for a patient with non-metastatic breast cancer comprises one or more of surgical removal of the primary tumor, surgical removal of the breast, radiation therapy, and neoadjuvant therapy. In some embodiments, the treatment for the patient with non-metastatic breast cancer excludes adjuvant therapy.

In some embodiments of the above-disclosed aspects, the method further comprises measuring the expression level of one or more of RKIP, HMGA2, CCL5, and TAM-metagene in a biological sample from the patient. In some embodiments, the method further comprises comparing the expression level of one or more of RKIP, HMGA2, CCL5, and TAM-metagene in a biological sample from the patient to the expression level of the same gene in a control non-metastatic tissue sample. In some embodiments, the patient is treated for metastatic breast cancer after the patient is determined to have at least three of: an elevated expression level of HMGA2, an elevated expression level of CCL5, an elevated expression level of TAM-metagene and a reduced expression level of RKIP in a biological sample from the patient relative to the expression level of the same genes in a control non-metastatic tissue sample.

A further aspect relates to a method for predicting a patient's prognosis for survival and/or metastasis-free survival of breast cancer comprising: measuring the expression level of one or more of RKIP, HMGA2, CCL5, and TAM-metagene in a biological sample from the patient; comparing the expression level of the one or more of RKIP, HMGA2, CCL5, TNFR2, GRN, and CCL7 in the biological sample from the patient to the expression level of the same gene or genes in a control non-metastatic tissue sample; predicting that the patient is likely to survive and or have metastatic-free survival when the measured level of one or more of HMGA2, CCL5, and TAM-metagene is reduced or not substantially different and/or the measured level of RKIP is elevated or not substantially different in a biological sample from the patient relative to the expression level of the same genes in a control non-metastatic tissue sample; and predicting that the patient is not likely to survive or have metastatic-free survival when the measured level of one or more of HMGA2, CCL5, and TAM-metagene is elevated and/or the measured level of RKIP is reduced in a biological sample from the patient relative to the expression level of the same genes in a control non-metastatic tissue sample.

In some embodiments, the patient is predicted to not likely survive or have metastatic-free survival after the patient is determined to have at least three of: an elevated expression level of HMGA2, an elevated expression level of CCL5, an elevated expression level of TAM-metagene and a reduced expression level of RKIP in a biological sample from the patient relative to the expression level of the same genes in a control non-metastatic tissue sample.

In some embodiments of the above disclosed aspects, the patient is determined to have a triple negative (ER⁻/PR⁻/HER2⁻) breast cancer (TNBC) subtype. In some embodiments of the above disclosed aspects, the method further comprising determining whether the breast cancer sample has a triple negative breast cancer (TNBC) subtype. In some embodiments of the above disclosed aspects, the biological sample from the patient is cancerous. In some embodiments of the above disclosed aspects, the biological sample from the patient comprises breast cancer tumor cells. In some embodiments of the above disclosed aspects, the control non-metastatic tissue sample comprises non-cancerous cells from the breast of the patient or from breast tissue that is not from the patient. In some embodiments of the above disclosed aspects, the control non-metastatic tissue sample comprises a reference level of expression from breast cancer tumors that are non-metastatic. In some embodiments of the above disclosed aspects, the method further comprises assaying nucleic acids in the breast cancer sample. In some embodiments, assaying nucleic acids comprises using PCR, microarray analysis, next generation RNA sequencing, or a combination thereof. In some embodiments of the above disclosed aspects, the method further comprises testing protein expression in the breast sample. In some embodiments, testing protein expression comprises performing ELISA, RIA, FACS, dot blot, Western Blot, immunohistochemistry, antibody-based radioimaging, mass spectroscopy, or a combination thereof. Embodiments may include measuring the level of expression of one or more genes. It is further understood that in alternative embodiments, the expression level of one or more biomarker genes may involve comparing the expression level of a control metastatic tissue sample having metastatic breast cancer cells. In this situation, the comparison would determine whether the expression level of the patient was similar from a statistically relevant perspective to the metastatic control.

In some embodiments of the above disclosed aspects, the method further comprises recording the expression level or the prognosis score in a tangible medium. In some embodiments, the method further comprises reporting the expression level or the prognosis score to the patient, a health care payer, a physician, an insurance agent, or an electronic system. In some embodiments, the method further comprises monitoring the patient for breast cancer recurrence or metastasis or prescribing a treatment that excludes the previously prescribed treatment.

Further aspects relate to a method of treating a patient determined to have metastatic breast cancer, comprising: administering a treatment that inhibits or reduces the expression level or protein activity of one or more of RKIP, HMGA2, CCL5, TNFR2, GRN, and CCL7. In some embodiments, the treatment inhibits or reduces the expression level or protein activity of CCL5. In some embodiments, the treatment comprises maraviroc. In some embodiments, the treatment comprises an EGFR inhibitor. In some embodiments, the EGFR inhibitor is tarceva. In some embodiments, the treatment inhibits or reduces the expression level or protein activity of GRN. In some embodiments, the treatment comprises an anti-GRN antibody. In some embodiments, the treatment inhibits or reduces the expression level or protein activity of CCL7. In some embodiments, the patient is determined to have a reduced expression level or protein activity of one or more of RKIP, HMGA2, CCL5, TNFR2, GRN, and CCL7.

In certain embodiments of the above-disclosed aspects, the act of determining an expression level or protein activity level comprises measuring the expression level or protein activity in a biological sample.

Certain methods may involve the use of a normalized sample or control that is based on one or more breast cancer samples that are not from the patient being tested. Methods may also involve obtaining a biological sample comprising breast cancer cells from the patient or obtaining a breast cancer sample.

Methods may further comprise assaying nucleic acids or testing protein expression in the breast cancer sample. In some embodiments, assaying nucleic acids comprises the use of PCR, microarray analysis, next generation RNA sequencing, any methods known in the art, or a combination thereof. In further embodiments, testing protein expression comprises ELISA, RIA, FACS, dot blot, Western Blot, immunohistochemistry, antibody-based radioimaging, mass spectroscopy, any methods known in the art, or a combination thereof.

Further aspects relate to a method of treating metastatic breast cancer in a patient in need thereof, the method comprising: administering a treatment that inhibits or reduces the expression level or protein activity of one or more of RKIP, HMGA2, CCL5, TNFR2, GRN, and CCL7. In some embodiments, the treatment inhibits or reduces the expression level or protein activity of CCL5, GRN, or both of CCL5 and GRN.

In some embodiments, the treatment that reduces the expression level or protein activity of CCL5 comprises maraviroc. In some embodiments, the treatment that reduces the expression level or protein activity of CCL5 comprises an EGFR inhibitor. In some embodiments, the EGFR inhibitor is tarceva.

In some embodiments, the treatment further comprises a second therapeutic agent. In some embodiments, the second therapeutic agent is a bromodomain inhibitor. The bromodomain may be a bromodomain protein known in the art or described herein. In some embodiments, the bromodomain comprises a protein listed in Table 1. In some embodiments, the bromodomain inhibitor comprises JQ1, I-BET 151 (GSK1210151A), I-BET 762 (GSK525762), OTX-015, TEN-010 (Tensha therapeutics), CPI-203, CPI-0610, RVX-208 (Resverlogix Corp), LY294002, or combinations thereof.

In some embodiments, the treatment and/or inhibitor is a small molecule inhibitor, a polypeptide inhibitor, an antagonistic antibody, or a nucleic acid inhibitor.

In some embodiments, the treatment inhibits or reduces the expression level or protein activity of GRN. In some embodiments, the treatment is an anti-GRN antibody. In some embodiments, the antibody is an A23 antibody. In some embodiments, the treatment comprises a small molecule inhibitor, a nucleic acid inhibitor, or a polypeptide inhibitor.

In some embodiments, the subject is diagnosed with or determined to have metastatic breast cancer. In some embodiments, the subject is or was determined to have a reduced expression level of RKIP and/or an elevated expression level of one or more of HMGA2, CCL5, and TAM-metagene. In some embodiments, the expression level is elevated or reduced relative to a control level of expression, such as a non-metastatic breast cancer or non-cancerous tissue. In some embodiments, the control level is a mean, an average, a normalized value, or a cut-off value. In some embodiments, the control level of expression is the level of expression in non-metastatic breast cancer. In some embodiments, the control level of expression is the level of expression in non-cancerous tissue. In some embodiments, the control is a cancerous tissue or a metastatic breast cancer tissue, and one skilled in the art would understand that a patient would be predicted to have metastatic breast cancer when the expression level of the measured genes in the patient sample is the same, or not significantly different, or within 1 or 2 standard deviations from a control that represents a level in metastatic breast cancer tissues.

In some embodiments, the subject is or was determined to have an elevated expression level of CCL5. In some embodiments, the subject is or was determined to have an elevated expression level of GRN.

In some embodiments, the expression or activity level of a protein is determined or has been from a biological sample from a patient or a control. In certain embodiments the sample is obtained from a biopsy from the breast tissue by any of the biopsy methods described herein or known in the art. In other embodiments the sample may be obtained from any of the tissues provided herein that include but are not limited to gall bladder, skin, heart, lung, pancreas, liver, muscle, kidney, smooth muscle, bladder, intestine, brain, prostate, esophagus, or thyroid tissue. Alternatively, the sample may include but not be limited to blood, serum, sweat, hair follicle, buccal tissue, tears, menses, urine, feces, or saliva. In particular embodiments, the sample may be a tissue sample, a whole blood sample, a urine sample, a saliva sample, a serum sample, a plasma sample or a fecal sample. In some embodiments, the biological sample may be from a tumor, a cyst, or neoplastic tissue.

In some embodiments the method further comprises comparing the expression level or protein activity of one or more of RKIP, HMGA2, CCL5, TNFR2, GRN, and CCL7 in a sample from the subject to the expression level or protein activity of one or more of RKIP, HMGA2, CCL5, TNFR2, GRN, and CCL7 in a control sample.

In further embodiments, the sample may be a fresh, frozen or preserved sample or a fine needle aspirate. In particular embodiments, the sample is a formalin-fixed, paraffin-embedded (FFPE) sample. An acquired sample may be placed in short term or long term storage by placing in a suitable medium, excipient, solution, or container. In certain cases storage may require keeping the sample in a refrigerated, or frozen environment. The sample may be quickly frozen prior to storage in a frozen environment. In certain instances the frozen sample may be contacted with a suitable cryopreservation medium or compound. Examples of cryopreservation mediums or compounds include but are not limited to: glycerol, ethylene glycol, sucrose, or glucose.

Some embodiments further involve isolating nucleic acids such as ribonucleic or RNA from a biological sample or in a sample of the patient. Other steps may or may not include amplifying a nucleic acid in a sample and/or hybridizing one or more probes to an amplified or non-amplified nucleic acid. The methods may further comprise assaying nucleic acids in a sample. Further embodiments include isolating or analyzing protein expression in a biological sample for the expression of polypeptides described herein, such as RKIP, HMGA2, CCL5, GRN, and TAM-metagene.

In certain embodiments, a microarray may be used to measure or assay the level of protein expression in a sample. The methods may further comprise recording the expression or activity level in a tangible medium or reporting the expression or activity level to the patient, a health care payer, a physician, an insurance agent, or an electronic system.

In some embodiments, methods will involve determining or calculating a prognosis score based on data concerning the expression or activity level of one or more genes, meaning that the expression or activity level of a gene is at least one of the factors on which the score is based. A prognosis score will provide information about the patient, such as the general probability whether the patient is sensitive to a particular therapy or has poor survival or high chances of recurrence. In certain embodiments, a prognosis value is expressed as a numerical integer or number that represents a probability of 0% likelihood to 100% likelihood that a patient has a chance of poor survival or cancer recurrence or poor response to a particular treatment.

In some embodiments, the prognosis score is expressed as a number that represents a probability of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% likelihood (or any range derivable therein) that a patient has a chance of poor survival or cancer recurrence or poor response to a particular treatment. Alternatively, the probability may be expressed generally in percentiles, quartiles, or deciles.

A difference between or among weighted coefficients or expression or activity levels or between or among the weighted comparisons may be, be at least or be at most about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 times or -fold (or any range derivable therein).

In some embodiments, determination of calculation of a diagnostic, prognostic, or risk score is performed by applying classification algorithms based on the expression values of biomarkers with differential expression p values of about, between about, or at most about 0.005, 0.006, 0.007, 0.008, 0.009, 0.01, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.1, 0.2, 0.3, 0.4, 0.5, 0.6, 0.7, 0.8, 0.9 or higher (or any range derivable therein). In certain embodiments, the prognosis score is calculated using one or more statistically significantly differentially expressed biomarkers (either individually or as difference pairs), including expression or activity levels in a gene or protein.

Any of the methods described herein may be implemented on tangible computer-readable medium comprising computer-readable code that, when executed by a computer, causes the computer to perform one or more operations. In some embodiments, there is a tangible computer-readable medium comprising computer-readable code that, when executed by a computer, causes the computer to perform operations comprising: a) receiving information corresponding to an expression or activity level of a gene or protein in a sample from a patient; and b) determining a difference value in the expression or activity levels using the information corresponding to the expression or activity levels in the sample compared to a control or reference expression or activity level for the gene.

In other aspects, tangible computer-readable medium further comprise computer-readable code that, when executed by a computer, causes the computer to perform one or more additional operations comprising making recommendations comprising: wherein the patient in the step a) is under or after a first treatment for colorectal cancer, administering the same treatment as the first treatment to the patient if the patient does not have increased expression or activity level; administering a different treatment from the first treatment to the patient if the patient has increased expression or activity level.

In some embodiments, receiving information comprises receiving from a tangible data storage device information corresponding to the expression or activity levels from a tangible storage device. In additional embodiments the medium further comprises computer-readable code that, when executed by a computer, causes the computer to perform one or more additional operations comprising: sending information corresponding to the difference value to a tangible data storage device, calculating a prognosis score for the patient, treating the patient with a traditional colorectal therapy if the patient does not have expression or activity levels, and/or or treating the patient with an alternative colorectal therapy if the patient has increased expression or activity levels.

The tangible, computer-readable medium further comprise computer-readable code that, when executed by a computer, causes the computer to perform one or more additional operations comprising calculating a prognosis score for the patient. The operations may further comprise making recommendations comprising: administering a treatment comprising a thymidylate synthase inhibitor to a patient that is determined to have a decreased expression or activity level.

Use of the one or more compositions may be employed based on methods described herein. Other embodiments are discussed throughout this application. Any embodiment discussed with respect to one aspect of the invention applies to other aspects of the invention as well and vice versa. The embodiments in the Example section are understood to be embodiments o that are applicable to all aspects of the technology described herein.

It is contemplated that any method or composition described herein can be implemented with respect to any other method or composition described herein and that different embodiments may be combined.

Any embodiment of the disclosure relating to a polypeptide or nucleic acid is contemplated also to cover embodiments whose sequences are at least 80, 81, 82, 83, 84, 85, 86, 87, 88, 89, 90, 91, 92, 93, 94, 95, 96, 97, 98, 99% identical to the polypeptide or nucleic acid.

The term “recurrence” refers to the detection of breast cancer in the form of metastatic spread of tumor cells, local recurrence, contralateral recurrence or recurrence of breast cancer at any site of the body of the patient after breast cancer had been substantially undetectable or responsive to treatments.

The term “metastasis,” as used herein, refers to the condition of spread of cancer from the organ of origin to additional distal sites in the patient. The process of tumor metastasis is a multistage event involving local invasion and destruction of intercellular matrix, intravasation into blood vessels, lymphatics or other channels of transport, survival in the circulation, extravasation out of the vessels in the secondary site and growth in the new location (Fidler et al., 1978; Liotta et al., 1988; Nicolson, 1988; and Zetter, 1990). Increased malignant cell motility has been associated with enhanced metastatic potential in animal as well as human tumors (Hosaka et al., 1978 and Haemmerlin et al., 1981).

“Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

“Subject” or “patient” refers to any single subject for which therapy is desired, including humans, cattle, dogs, guinea pigs, rabbits, chickens, and so on. Also intended to be included as a subject are any subjects involved in clinical research trials not showing any clinical sign of disease, or subjects involved in epidemiological studies, or subjects used as controls.

“Remission” refers to a period during which symptoms of disease are reduced (partial remission) or disappear (complete remission). With regard to cancer, remission means there is no sign of it on scans or medical examination. “Remission” is used instead of cure regarding cancer because it cannot be sure that there are no cancer cells at all in the body. So the cancer could recur in the future, although there is no sign of it at the time. More specifically, “remission” could mean the tumor-free time period, and is dated from the first, not the last, therapy session. Patients with tumors that recur within one month of treatment ending are considered to have had no remission. Disappearance of all disease is complete remission; reduction tumor size by more than 50 percent is considered partial remission.

By “gene” is meant any polynucleotide sequence or portion thereof with a functional role in encoding or transcribing a protein or regulating other gene expression. The gene may consist of all the nucleic acids responsible for encoding a functional protein or only a portion of the nucleic acids responsible for encoding or expressing a protein. The polynucleotide sequence may contain a genetic abnormality within exons, introns, initiation or termination regions, promoter sequences, other regulatory sequences or unique adjacent regions to the gene.

As used herein, “treatment” or “therapy” is an approach for obtaining beneficial or desired clinical results. This includes: reduce the number of cancer cells; reduce the tumor size; inhibit (i.e., slow to some extent and/or stop) cancer cell infiltration into peripheral organs; inhibit (i.e., slow to some extent and/or stop) tumor metastasis; inhibit, to some extent, tumor growth; and/or relieve to some extent one or more of the symptoms associated with the disorder, shrinking the size of the tumor, decreasing symptoms resulting from the disease, increasing the quality of life of those suffering from the disease, decreasing the dose of other medications required to treat the disease, delaying the progression of the disease, and/or prolonging survival of patients.

The term “therapeutically effective amount” refers to an amount of the drug that may reduce the number of cancer cells; reduce the tumor size; inhibit (i.e., slow to some extent and particularly stop) cancer cell infiltration into peripheral organs; inhibit (i.e., slow to some extent and particularly stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “elevated expression,” “overexpress”, “overexpression”, “overexpressed”, “up-regulate”, or “up-regulated” interchangeably refer to a biomarker that is transcribed or translated at a detectably greater level, usually in a cancer cell, in comparison to a non-cancer cell or cancer cell that is not associated with the worst or poorest prognosis. The term includes overexpression due to transcription, post transcriptional processing, translation, post-translational processing, cellular localization, and/or RNA and protein stability, as compared to a non-cancer cell or cancer cell that is not associated with the worst or poorest prognosis. Overexpression can be detected using conventional techniques for detecting mRNA (i.e., RT-PCR, PCR, hybridization) or proteins (i.e., ELISA, immunohistochemical techniques, mass spectroscopy). Overexpression can be 10%, 20%, 30%, 40%, 50%, 60%, 70%, 80%, 90% or more (or any range derivable therein) in comparison to a normal cell or cancer cell that is not associated with the worst or poorest prognosis. In certain instances, overexpression is 1-fold, 2-fold, 3-fold, 4-fold 5, 6, 7, 8, 9, 10, or 15-fold or more higher levels of transcription or translation (or any range derivable therein) in comparison to a non-cancer cell or cancer cell that is not associated with the worst or poorest prognosis. The comparison may be a direct comparison where the expression level of a control is measured at the same time as the test sample or it may be a level of expression that is determined from a previously evaluated sample or an average of levels of expression of previously evaluated sample(s). As mentioned above, a patient may be determined to have a relative level of expression by comparing the level of expression of a biomarker to a non-metastatic control or to a metastatic control. If the latter, the expression may not be elevated or reduced, but within 1, 2, 3, 4, or 5 standard deviation(s) (or any range derivable therein) of the expression level for that biomarker.

“Biological sample” includes sections of tissues such as biopsy and autopsy samples, and frozen sections taken for histologic purposes. Such samples include breast cancer tissues, cultured cells, e.g., primary cultures, explants, and transformed cells. A biological sample is typically obtained from a mammal, such as a primate, e.g., human. The biological sample, in some embodiments, may include metastatic tissue.

A “biopsy” refers to the process of removing a tissue sample for diagnostic or prognostic evaluation, and to the tissue specimen itself. Any biopsy technique known in the art can be applied to the diagnostic and prognostic methods. The biopsy technique applied will depend on the tissue type to be evaluated (e.g., breast), the size and type of the tumor, among other factors. Representative biopsy techniques include, but are not limited to, excisional biopsy, incisional biopsy, needle biopsy, and surgical biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy”, or a “fine-needle aspiration biopsy” which generally obtains a suspension of cells from within a target tissue. Biopsy techniques are discussed, for example, in Harrison's Principles of Internal Medicine, 2005. Obtaining a biopsy includes both direct and indirect methods, including obtaining the biopsy from the patient or obtaining the biopsy sample after it is removed from the patient.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-C: Non-metastatic RKIP+ tumors contain fewer macrophages. A) Quantile-quantile (qq)-plot showing ranked −log 10 transformed p-values among macrophagespecific genes (y-axis) relative to a similar bootstrapped distribution in blue (x-axis). Distortion of the density above x=y (red line) indicates that the measured p-values are systematically lower than expected by chance. B) Quantitation of relative macrophage presence in xenograft (BM1 and MDA-MB-436) and syngeneic (4T1.2) tumor models with and without the expression of RKIP. Infiltration was quantified as the proportion of Inner tumor mass positively stained with F4/80 via immunohistochemistry. C) Representative images of relative macrophage presence in xenograft (BM1 and MDA-MB-436) and syngeneic (4T1.2) tumor models with and without RKIP expression were sectioned and immunostained for F4/80. *0.05<p<0.01 **0.01≤p<0.001 ***0.001≤p

FIG. 2A-F: RKIP suppresses recruitment of a distinct TAM population that potentiates tumor cell invasion. A) In this experiment BM1 or BM1+RKIP cells were pretreated with conditioned media from the human THP-1 monocyte cell line for 24 hrs. Media contained 10% serum and was grown for 24 hrs with approximately 1 million cells per mL of media. Cells were plated in transwell inserts coated with matrigel, and invaded cells were stained with Calcein and measured after 24 hours. Relative invasion is calculated by the amount of Calcein fluorescence normalized to control cell invasion. P-values were calculated using an unpaired T-test with Welch's correction, N=8 per group. B) BM1 or BM1+RKIP cells were pretreated with conditioned media from TAMs (BM1 TAMs or BM1+RKIP TAMs) for 24 hours prior to performing invasion assays. Relative invasion was calculated as above compared using cells treated with serum free media as control. P-values were calculated using an unpaired T-test with Welch's correction, N=5. C) BM1 cells were pretreated with TAM conditioned media (BM1 or BM1+RKIP TAMs) for 24 hours prior to invasion assays, using TAMs from four independent tumors each. Invasion assays were done as described before and normalized to serum free treated BM1 cells. P-values were obtained using an unpaired T-test with Welch's correction, N=6. D) TAM conditioned media from four independent tumors each was analyzed for expression using RayBiotech L308 Mouse Cytokine Arrays. Relative abundance for RKIP derived TAMs were normalized to control tumor TAMs. Shown are the proteins with 0.8 or lower relative abundance E) Relative expression for RKIP derived TAMs were normalized to control tumor TAMs. Shown are the proteins with >1.2 relative expression. F) Relative mRNA was measured from three independent TAM samples per group. Relative mRNA was calculated as compared to control TAMs, with Gapdh as the reference gene. P-values were calculated between Control and RKIP derived TAMs using a student's T-test. *0.05<p<0.01 **0.01≤p<0.001 ***0.001≤p

FIG. 3A-H: Overexpression of CCL5 restores TAMs and overcomes metastasis suppression in RKIP+ tumors. A) Gene Set Enrichment Analysis identifies enrichment of genes involved in cytokine-cytokine receptor interactions (black lines) among genes with anticorrelated expression levels in tumors and surrounding stroma. Genes are ranked on the x-axis by the estimated spearman correlation of homolog pair expression levels in tumors and surrounding stroma (gray curve and color bars), and the running enrichment score is indicated on the y-axis (green curve). B) Tumor genes differentially expressed in RKIP tumors relative to Control (p<0.05) are shown from the external stimulus (GO) category from our RNAseq data. C) qRT-PCR was performed on mRNA purified from xenograft tumors (BM1) that do (Non-invasive) and do not (Invasive) express exogenous RKIP. Species specific primers were used to detect relative mRNA abundance of CCL5 (Hs) and Ccr5 (Mm) in the tumor and stroma, respectively; bars indicate the mean expression level (+s.e.m.). Relative mRNA was calculated using 2^{-ΔΔCT} normalized to GAPDH (Hs) or Rpl4 (Mm); expression levels observed in invasive samples was set to 1 by construction. D) Relative mRNA was calculated using 2^{-ΔΔCT} of BM1+RKIP TAMs compared to BM1 TAMs with Gapdh as the reference gene. E) Representative images of relative macrophage presence in xenograft BM1 tumors with and without RKIP and CCL5 expression. F) Estimated relative macrophages (y-axis) in BM1 (left bars) and BM1+RKIP (right bars) with or without exogenous CCL5 expression in tumors (grey and white, respectively). Infiltration was quantified as the proportion of total tumor area positively stained with F4/80; n=3 mice in each group. G) BM1 cells were pretreated with TAM conditioned media (BM1, BM1+RKIP, BM1+RKIP+CCL5, or BM1+CCL5 TAMs) for 24 hours prior to invasion assays, using TAMs from four independent tumors each. Invasion assays were done as described before and normalized to serum free treated BM1 cells. P-values were obtained using an unpaired T-test with Welch's correction, N=6. H) Relative abundance of tumor-derived cells in BM1 in xenograft mouse models (n=4 in each model). Relative tumor cell abundance in isolated blood cells was estimated 4 weeks following injection as the ratio of GAPDH/Gapdh transcripts derived from human (tumor) or mouse tissue by qRTPCR. Significance in all panels was estimated via a Student's T-test. *0.05<p<0.01 **0.01≤p<0.001 ***0.001≤p

FIG. 4A-D: RKIP blocked TAM phenotype rescued by CCL5. A) TAM conditioned media from four independent tumors each was analyzed for expression using RayBiotech L308 Mouse Cytokine Arrays. RKIP+CCL5 derived TAMs were normalized to RKIP derived TAMs with a cutoff set at greater than 3-fold expression. B) Relative protein is shown for each gene from the Cytokine array compared to BM1 derived TAMs for each gene. C) Relative mRNA was measured from three independent TAM samples per group. Relative mRNA was calculated as compared to control TAMs, with Gapdh as the reference gene. Pvalues were calculated between RKIP and RKIP+CCL5 derived TAMs using a student's T-test. D) Protein levels (spectral counts) from mass-spec data is shown for genes in monocytes (MO), LPS induced macrophages (M1), and IL-4 induced macrophages (M2). P-values were calculated using a Student's T-test. *0.05<p<0.01 **0.01≤p<0.001 ***0.001≤p

FIG. 5A-D: Suppression of metastasis and TAMs by RKIP is coordinated though HMGA2 Signaling. A) BM1 cells were transduced independently with two separate shRNAs targeting HMGA2. Relative expression of HMGA2 and CCL5 was compared to control shRNA transduced cells and normalized to internal GAPDH expression. Values were obtained from triplicate biological samples. P-values were calculated using a student's T-test. B) qRT-PCR using mRNA purified from wild type and Hmga2−/− knockout mice (n=4). Expression of each gene is shown relative to estimates derived from wild type Hmga2+/+ cells using the ΔΔCT method relative to Gapdh expression. C) Representative images of macrophage infiltration in wild type and Hmga2 knockout mice as determined through F4/80+ staining. Stroma regions are delimited by the dashed lines. B) quantitation of macrophage infiltration in wild type Hmga2+/+ and Hmga2−/− knockout mice for both tumor and in stroma regions. F4/80+ cells were quantified by counting 5 high power fields in each section. D) A schematic showing the regulation of CCL5 by RKIP through HMGA2. Previous publications from our group have demonstrated that RKIP regulates HMGA2 through a Let-7 dependent model through inhibition of Raf. *0.05<p<0.01 **0.01≤p<0.001 ***0.001≤p

FIG. 6A-D: An RKIP-HMGA2-CCL5-macrophage gene signature predicts metastasis-free Survival. A) Expression estimates of genes are from a set of 871 breast cancer patients, stratified into either TNBC or non-TNBC patients. SLPI, OPN, MMP12, CCL7, TNFR2, GRN, TMEFF1, and CCL5 expression levels we compared between TNBC and non-TNBC patients using a Student's T-test. B) Individual scores for each gene in comparison to CCL5 expression are plotted for 12 separate genes. C) Kaplan-Meier plots are shown for a set of all 871 breast cancer patients, those that are non-TNBC patients in the set (770), and those that are TNBC patients within the set (101). D) Heatmap identifying data sets (top) where breast cancer metastasis free survival is significantly stratified by classifier (right)*0.05<p<0.01 **0.01≤p<0.001 ***0.001≤p

FIG. 7. TNBC-TAM crosstalk. A schematic displaying the circular interplay between TNBC cells and TAMs. RKIP regulates CCL5 expression, which can recruit TAMs. TNFR2, GRN, CCL7 (all part of our macrophage metagene) are displayed as prominent pro-invasive TAM genes that act back on the TNBC cells.

FIG. 8: Validation of RKIP expression in MDA-MB-231 BM1 cells. Protein lysate from human bonetropic metastatic MDA-MB-231 derived BM1 (1833), MDA-MB-436 and mouse 4T1.2 triple negative breast cancer expressing pCDH1 vector or wild-type RKIP were immunoblotted with RKIP and α-tubulin antibodies.

FIG. 9A-B: A) Scheme depicting comparison between tumor and stroma-derived mRNA expression levels from isogenic TNBC xenograft models in which B) the metastatic phenotype is suppressed by exogenous expression of RKIP.

FIG. 10A-B: Gene expression in metastatic (A) and non-metastatic cells (B).

FIG. 11: 20,000 BM1 cells (control, RKIP, CCL5, or RKIP+CCL5 transduced) were plated in transwell inserts coated with matrigel, and invaded cells were stained with Calcein and measured after 24 hours. Relative invasion is calculated by the amount of Calcein fluorescence normalized to control cell invasion. P-values were calculated using an unpaired T-test with Welch's correction, N=8 per group. *0.05<p<0.01 **0.01≤p<0.001 *** 0.001≤p

FIG. 12: qRT-PCR was performed on mRNA purified from human (MDA-MB-436) and mouse (4T1.2) TNBC cell lines that do (RKIP) and do not (VC) express exogenous RKIP. Species specific primers were used to detect relative mRNA abundance of CCL5 (Hs) or Ccl5 (Mm) (+s.e.m.). Relative mRNA was calculated using 2-ΔΔCT normalized to GAPDH (Hs) or Gapdh (Mm).

FIG. 13: Protein Lysate was obtained from tumor samples using RIPA buffer by sonication. 20 ug of total protein was run per lane. In 1833 (also termed BM1) tumors that were control, RKIP overexpressing, CCL5 overexpressing, or RKIP+CCL5 overexpressing were immunoblotted for RKIP and CCL5 with Tubulin as a loading control. The blot shown is representative of four independent tumor groups.

FIG. 14: CCL5 levels were measured from media of cells cultured for 24 hours in SF media using a RayBiotech CCL5 ELISA. A standard curve was used to calculated amounts of protein from three independent experiments. P-values were calculated using a Student's T-Test. *0.05<p<0.01 **0.01≤p<0.001 ***0.001≤p

FIG. 15: Estimated relative macrophages (y-axis) in MDA-MB-436 (left bars) and MDA-MB-436+RKIP (right bars) with or without exogenous CCL5 expression in tumors (grey and white, respectively). Infiltration was quantified as the proportion of total tumor area positively stained with F4/80; n=3 mice in each group. *0.05<p<0.01 **0.01≤p<0.001 *** 0.001≤p

FIG. 16A-F: Expression estimates of genes are from a breast cancer patients, stratified into either TNBC or non-TNBC patients. SLPI, OPN (SPP1), MMP12, CCL7, TNFR2, GRN, LGALS3, TMEFF1, and CCL5 or PEBP1 (RKIP), HMGA2, CCL5 and CCR5 expression levels we compared between TNBC and non-TNBC patients using a Student's T-test.

FIG. 17: Individual scores for each gene in comparison to CCL5 expression are plotted for 12 separate genes across four data sets.

FIG. 18: Heatmap identifying data sets (left) where breast cancer metastasis free survival is significantly stratified by classifier (top).

FIG. 19: Tumor growth was measure twice per week using calipers. Tumor volum was calculated as (π/6)*width²*length. P-value was obtained using a 2-way ANOVA comparing the different tumor groups over time.

FIG. 20A-F: A) Final tumor weights shown for nude mice treated with the anti-Progranulin neutralizing antibody A23 alone or in combination with the CCR5 inhibitor Maraviroc for 21 days starting at day 3. B) The corresponding growth curves for the treated mice. Tumor volume is calculated as (π/6)*length2*width C) To measure the _rst step of metastasis we examine the relative number of tumor cells intravasating into the blood stream. This is calculated as the relative amount of human GAPDH compared to mouse Gapdh mRNA from cells isolated from blood after treatment. D) Tumor-associated macrophages were isolated from treated mice and qRT-PCR was performed to determine the relative amount of mRNA with Gapdh serving as the loading control; each set of four bars is data from the control, A23, Maraviroc, and A23+Maraviroc, respectively. E-F) Tumor cells were isolated from tumors stripped of mouse cells. RNA was collected and Relative CCL5 and Progranulin (GRN) were calculated with GAPDH as a loading control. *0.05>p>0.01 ** 0.01>p>0.001

FIG. 21: Relative levels of HMGA2 (first bar of each set) and CCL5 (second bar of each set) are shown for the human triple-negative BM1 cell line (top) and the mouse basal-like breast cancer M6C cell line (bottom) treated with the bromodomain inhibitor JQ1, known to block HMGA2 activity. Relative levels of mRNA were calculated using the formula RQ=2-^{˜˜}CT with GAPDH as the loading control*0.05>p>0.01 ***0.001>p.

### DETAILED DESCRIPTION

Although triple negative breast cancers (TNBC) are the most aggressive subtype of breast cancer, they currently lack targeted therapies. Because this classification still includes a heterogeneous collection of tumors, new tools to classify TNBCs are urgently required in order to improve the prognostic capability for high risk patients and predict response to therapy and/or to predict the prognosis for metastasis-free survival. Furthermore, identifying patients at higher risk for metastasis will allow for a more aggressive approach at an earlier timepoint, which provides for improved treatment methods for individuals with metastatic breast cancer.

### I. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from colorectal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects the sample is obtained from cystic fluid or fluid derived from a tumor or neoplasm. In yet other embodiments the cyst, tumor or neoplasm is breast tissue. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, scraping, phlebotomy, or any other methods known in the art. In some cases, the sample may be obtained, stored, or transported using components of a kit of the present methods. In some cases, multiple samples, such as multiple colorectal samples may be obtained for diagnosis by the methods described herein. In other cases, multiple samples, such as one or more samples from one tissue type (for example breast) and one or more samples from another tissue may be obtained for diagnosis by the methods. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a colorectal or a suspected colorectal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, proteins, polypeptides, genes, gene fragments, expression products, gene expression products, protein expression products or fragments, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### II. Analysis of Gene Expression

In certain aspects a meta-analysis of expression or activity can be performed. In statistics, a meta-analysis combines the results of several studies that address a set of related research hypotheses. This is normally done by identification of a common measure of effect size, which is modeled using a form of meta-regression. Generally, three types of models can be distinguished in the literature on meta-analysis: simple regression, fixed effects meta-regression and random effects meta-regression. Resulting overall averages when controlling for study characteristics can be considered meta-effect sizes, which are more powerful estimates of the true effect size than those derived in a single study under a given single set of assumptions and conditions. A meta-gene expression value, in this context, is to be understood as being the median of the normalized expression of a marker gene or activity. Normalization of the expression of a marker gene may be achieved by dividing the expression level of the individual marker gene to be normalized by the respective individual median expression of this marker genes, wherein said median expression may be calculated from multiple measurements of the respective gene in a sufficiently large cohort of test individuals. The test cohort may comprise at least 3, 10, 100, 200, 1000 individuals or more including all values and ranges thereof. Dataset-specific bias can be removed or minimized allowing multiple datasets to be combined for meta-analyses (See Sims et al. BMC Medical Genomics (1:42), 1-14, 2008, which is incorporated herein by reference in its entirety).

The calculation of a meta-gene expression value is performed by: (i) determining the gene expression value of at least two, or more genes (ii) “normalizing” the gene expression value of each individual gene by dividing the expression value with a coefficient which is approximately the median expression value of the respective gene in a representative breast cancer cohort (iii) calculating the median of the group of normalized gene expression values.

A gene shall be understood to be specifically expressed in a certain cell type if the expression level of said gene in said cell type is at least 2-fold, 5-fold, 10-fold, 100-fold, 1000-fold, or 10000-fold higher than in a reference cell type, or in a mixture of reference cell types. Reference cell types include non-cancerous breast tissue cells or a heterogeneous population of breast cancers.

Comparison of multiple marker genes with a threshold level can be performed as follows: 1. The individual marker genes are compared to their respective threshold levels. 2. The number of marker genes, the expression level of which is above their respective threshold level, is determined. 3. If a marker genes is expressed above its respective threshold level, then the expression level of the marker gene is taken to be “above the threshold level”.

In certain aspects, the determination of expression levels is on a gene chip, such as an Affymetrix™ gene chip. In another aspect, the determination of expression levels is done by kinetic real time PCR.

In certain aspects, the methods can relate to a system for performing such methods, the system comprising (a) apparatus or device for storing data on the receptors status (ER, AR, or PR, GR) or nodal status of the patient; (b) apparatus or device for determining the expression level of at least one marker gene or activity; (c) apparatus or device for comparing the expression level of the first marker gene or activity with a predetermined first threshold value; (d) apparatus or device for determining the expression level of at least one second, third, fourth, 5^{th}, 6^{th} or more marker gene or activity and for comparing with a corresponding predetermined threshold; and (e) computing apparatus or device programmed to provide a unfavorable or poor prognosis or favorable prognosis based on the comparisons.

The person skilled in the art readily appreciates that an unfavorable or poor prognosis can be given if the expression level of the first marker gene with the predetermined first threshold value indicates a tumor that is likely to recur or not respond well to standard therapies.

The expression patterns can also be compared by using one or more ratios between the expression levels of different breast cancer biomarkers. Other suitable measures or indicators can also be employed for assessing the relationship or difference between different expression patterns.

The expression levels of breast cancer biomarkers can be compared to reference expression levels using various methods. These reference levels can be determined using expression levels of a reference based on all breast cancer patients. Alternatively, it can be based on an internal reference such as a gene that is expressed in all cells. In some embodiments, the reference is a gene expressed in breast cancer cells at a higher level than any biomarker. Any comparison can be performed using the fold change or the absolute difference between the expression levels to be compared. One or more breast cancer biomarkers can be used in the comparison. It is contemplated that 1, 2, 3, 4, 5, 6, 7, 8, 9, 10, and/or 11 biomarkers (or any range derivable therein) may be compared to each other and/or to a reference that is internal or external. A person of ordinary skill in the art would know how to do such comparisons.

Comparisons or results from comparisons may reveal or be expressed as x-fold increase or decrease in expression relative to a standard or relative to another biomarker or relative to the same biomarker but in a different class of prognosis. In some embodiments, patients with a poor prognosis have a relatively high level of expression (overexpression) or relatively low level of expression (underexpression) when compared to patients with a better or favorable prognosis, or vice versa.

Fold increases or decreases may be, be at least, or be at most 1-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100- or more, or any range derivable therein. Alternatively, differences in expression may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000% difference, or any range derivable therein.

Other ways to express relative expression levels are with normalized or relative numbers such as 0.001, 0.002, 0.003, 0.004, 0.005, 0.006, 0.007, 0.008, 0.009, 0.01, 0.02, 0.03. 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein. In some embodiments, the levels can be relative to a non-metastatic control or relative to a metastatic control.

Algorithms, such as the weighted voting programs, can be used to facilitate the evaluation of biomarker levels. In addition, other clinical evidence can be combined with the biomarker-based test to reduce the risk of false evaluations. Other cytogenetic evaluations may be considered in some embodiments.

Any biological sample from the patient that contains breast cancer cells may be used to evaluate the expression pattern of any biomarker discussed herein. In some embodiments, a biological sample from a breast tumor is used. Evaluation of the sample may involve, though it need not involve, panning (enriching) for cancer cells or isolating the cancer cells.

### A. Measurement of Gene Expression Using Nucleic Acids

Testing methods based on differentially expressed gene products are well known in the art. In accordance with one aspect, the differential expression patterns of breast cancer biomarkers can be determined by measuring the levels of RNA transcripts of these genes, or genes whose expression is modulated by the these genes, in the patient's breast cancer cells. Suitable methods for this purpose include, but are not limited to, RT-PCR, Northern Blot, in situ hybridization, Southern Blot, slot-blotting, nuclease protection assay and oligonucleotide arrays.

In certain aspects, RNA isolated from breast cancer cells can be amplified to cDNA or cRNA before detection and/or quantitation. The isolated RNA can be either total RNA or mRNA. The RNA amplification can be specific or non-specific. Suitable amplification methods include, but are not limited to, reverse transcriptase PCR, isothermal amplification, ligase chain reaction, and Qbeta replicase. The amplified nucleic acid products can be detected and/or quantitated through hybridization to labeled probes. In some embodiments, detection may involve fluorescence resonance energy transfer (FRET) or some other kind of quantum dots.

Amplification primers or hybridization probes for a breast cancer biomarker can be prepared from the gene sequence or obtained through commercial sources, such as Affymatrix. In certain embodiments the gene sequence is identical or complementary to at least 8 contiguous nucleotides of the coding sequence.

Sequences suitable for making probes/primers for the detection of their corresponding breast cancer biomarkers include those that are identical or complementary to all or part of the breast cancer biomarker genes described herein. These sequences are all nucleic acid sequences of breast cancer biomarkers.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. In some embodiments, because each of the biomarkers has more than one human sequence, it is contemplated that probes and primers may be designed for use with each of these sequences. For example, inosine is a nucleotide frequently used in probes or primers to hybridize to more than one sequence. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In another embodiment, the probes/primers for a gene are selected from regions which significantly diverge from the sequences of other genes. Such regions can be determined by checking the probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by one of ordinary skill in the art.

In one embodiment, quantitative RT-PCR (such as TaqMan, ABI) is used for detecting and comparing the levels of RNA transcripts in breast cancer samples. Quantitative RT-PCR involves reverse transcription (RT) of RNA to cDNA followed by relative quantitative PCR (RT-PCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable cDNAs may be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes one or more internal PCR standards. The internal standard may be an abundant housekeeping gene in the cell or it can specifically be GAPDH, GUSB and (3-2 microglobulin. These standards may be used to normalize expression levels so that the expression levels of different gene products can be compared directly. A person of ordinary skill in the art would know how to use an internal standard to normalize expression levels.

A problem inherent in clinical samples is that they are of variable quantity and/or quality. This problem can be overcome if the RT-PCR is performed as a relative quantitative RT-PCR with an internal standard in which the internal standard is an amplifiable cDNA fragment that is similar or larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs.

Nucleic acid arrays can also be used to detect and compare the differential expression patterns of breast cancer biomarkers in breast cancer cells. The probes suitable for detecting the corresponding breast cancer biomarkers can be stably attached to known discrete regions on a solid substrate. As used herein, a probe is “stably attached” to a discrete region if the probe maintains its position relative to the discrete region during the hybridization and the subsequent washes. Construction of nucleic acid arrays is well known in the art. Suitable substrates for making polynucleotide arrays include, but are not limited to, membranes, films, plastics and quartz wafers.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be 50, 100, 200, 300, 400, 500 or more probes/cm².

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more breast cancer biomarkers with respect to diagnostic, prognostic, and treatment methods.

Certain embodiments may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448A1, all of which are specifically incorporated by reference.

In one embodiment, nuclease protection assays are used to quantify RNAs derived from the breast cancer samples. There are many different versions of nuclease protection assays known to those practiced in the art. The common characteristic that these nuclease protection assays have is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

### B. Measurement of Gene Expression Using Proteins and Polypeptides

In other embodiments, the differential expression patterns of breast cancer biomarkers can be determined by measuring the levels of polypeptides encoded by these genes in breast cancer cells. Methods suitable for this purpose include, but are not limited to, immunoassays such as ELISA, RIA, FACS, dot blot, Western Blot, immunohistochemistry, and antibody-based radioimaging. Protocols for carrying out these immunoassays are well known in the art. Other methods such as 2-dimensional SDS-polyacrylamide gel electrophoresis can also be used. These procedures may be used to recognize any of the polypeptides encoded by the breast cancer biomarker genes described herein.

One example of a method suitable for detecting the levels of target proteins in peripheral blood samples is ELISA. In an exemplifying ELISA, antibodies capable of binding to the target proteins encoded by one or more breast cancer biomarker genes are immobilized onto a selected surface exhibiting protein affinity, such as wells in a polystyrene or polyvinylchloride microtiter plate. Then, breast cancer cell samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Before being added to the microtiter plate, cells in the peripheral blood samples can be lysed using various methods known in the art. Proper extraction procedures can be used to separate the target proteins from potentially interfering substances.

In another ELISA embodiment, the breast cancer cell samples containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another typical ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control and/or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 49° C. overnight. Detection of the immunocomplex then requires a labeled secondary binding ligand or antibody, or a secondary binding ligand or antibody in conjunction with a labeled tertiary antibody or third binding ligand.

After all of the incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl)-benzhiazoline-6-sulfonic acid (ABTS) and hydrogen peroxide, in the case of peroxidase as the enzyme label. Quantitation can be achieved by measuring the degree of color generation, e.g., using a spectrophotometer.

Another suitable method is RIA (radioimmunoassay). An example of RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Various protocols for conducting RIA to measure the levels of polypeptides in breast cancer cell samples are well known in the art.

Suitable antibodies include, but are not limited to, polyclonal antibodies, monoclonal antibodies, chimeric antibodies, humanized antibodies, single chain antibodies, Fab fragments, and fragments produced by a Fab expression library.

Antibodies can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Protein array technology is discussed in detail in Pandey and Mann (2000) and MacBeath and Schreiber (2000), each of which is herein specifically incorporated by reference. These arrays typically contain thousands of different proteins or antibodies spotted onto glass slides or immobilized in tiny wells and allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as a breast cancer biomarker proteins.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

In other aspects, the levels of polypeptides in samples can be determined by detecting the biological activities associated with the polypeptides. If a biological function/activity of a polypeptide is known, suitable in vitro bioassays can be designed to evaluate the biological function/activity, thereby determining the amount of the polypeptide in the sample.

### III. Cancer Therapy

Certain embodiments are directed to methods of treating breast cancer based on the expression level of breast cancer biomarkers. Any known treatments that are contemplated for treating a triple negative breast cancer can be used (for example, see Andre et al., 2012, which is incorporated herein by reference in its entirety)

In certain aspects, there may be provided methods for treating a subject determined to have cancer and with a predetermined expression profile of one or more biomarkers disclosed herein.

In a further aspect, biomarkers and related systems that can establish a prognosis of cancer patients can be used to identify patients who may get benefit of conventional single or combined modality therapy. In the same way, those patients who do not get much benefit from such conventional single or combined modality therapy can be identified and can be offered alternative treatment(s).

In certain aspects, a first treatment regimen is administered to a patient having or predicted to have or develop metastatic breast cancer, and a second treatment regimen is administered to a patient having or predicted to have or develop non-metastatic breast cancer.

The first or second treatment regimen can include chemotherapy, therapeutic agents, hormonal therapy, surgical removal of the breast and/or ovaries, trastuzumab, and radiation therapy. In some aspects, the second treatment regimen (treatment for patients having or predicted to have non-metastatic breast cancer) excludes one or more of chemotherapy, therapeutic agents, hormonal therapy, surgical removal of the breast and/or ovaries, trastuzumab, and radiation therapy.

Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein transferase inhibitors, transplatinum, 5-fluorouracil, vincristin, vinblastin and methotrexate, or any analog or derivative variant of the foregoing.

Suitable therapeutic agents include, for example, vinca alkaloids, agents that disrupt microtubule formation (such as colchicines and its derivatives), anti-angiogenic agents, therapeutic antibodies, RKIP pathway targeting agents, tyrosine kinase targeting agent (such as tyrosine kinase inhibitors), serine kinase targeting agents, transitional metal complexes, proteasome inhibitors, antimetabolites (such as nucleoside analogs), alkylating agents, platinum-based agents, anthracycline antibiotics, topoisomerase inhibitors, macrolides, therapeutic antibodies, retinoids (such as all-trans retinoic acids or a derivatives thereof); geldanamycin or a derivative thereof (such as 17-AAG), and other standard chemotherapeutic agents well recognized in the art.

Certain chemotherapeutics are well known for use against breast cancer. These breast cancer chemotherapeutics are capecitabine, carboplatin, cyclophosphamide (Cytoxan), daunorubicin, docetaxel (Taxotere), doxorubicin (Adriamycin), epirubicin (Ellence), fluorouracil (also called 5-fluorouracil or 5-FU), gemcitabine, eribulin, ixabepilone, methotrexate, mitomycin C, mitoxantrone, paclitaxel (Taxol), albumin-bound paclitaxel, thiotepa, vincristine, liposomal doxorubicin, vinorelbine.

In some embodiments, the chemotherapeutic agent is any of (and in some embodiments selected from the group consisting of) aromatase inhibitor, toremifene, magestrol acetate, fluvestran, trastuzumab, docetaxel, liposomal doxorubicin, ixabepilone, albumin-bound paclitaxel, eribulin, adriamycin, colchicine, cyclophosphamide, actinomycin, bleomycin, daunorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Herceptin®, vinorelbine, capecitabine, Gleevec®, Alimta®, Avastin®, Velcade®, Tarceva®, Neulasta®, Lapatinib, STI-571, ZD1839, Iressa® (gefitinib), SH268, genistein, CEP2563, SU6668, SU11248, EMD121974, and Sorafenib.

In some embodiments, the aromatase inhibitor comprises one or more of letrozole, anastrozole, and exemstane

In some embodiments, the treatment regimen is a combination of the one or more chemotherapeutic agents described herein. In some embodiments, the treatment regimen excludes one or more of the chemotherapeutic agents described herein.

In some embodiments, the treatment regimen further comprises a bromodomain inhibitor. Recruitment of proteins to macromolecular complexes by acetylated lysine residues is mediated by bromodomains (BRDs), which are evolutionarily highly conserved protein-interaction modules that recognise ε-N-lysine acetylation motifs. Exemplary bromodomain proteins are shown below in Table 1:

| Protein | Name | Function | BRDs |
|---|---|---|---|
| ASH1L | Absent, small or homeotic-like | Methyltransferase | 1 |
| ATAD2A/B | AAA domain-containing protein 2 | ATPase, coactivator | 1 |
| BAZ1A/B | BRD adjacent to zinc finger domain | Chromatin assembly and | 1 |
| | protein 1A | remodelling | |
| BAZ2A/B | BRD adjacent to zinc finger domain | Unknown | 1 |
| | protein 2A/B | | |
| BRD1 | BRD-containing 1 | Transcription factor | 1 |
| BRD2 | BRD-containing 2 | Transcription factor | 2 |
| BRD3 | BRD-containing 3 | Transcription factor | 2 |
| BRD4 | BRD-containing 4 | Transcription factor | 2 |
| BRDT | BRD-containing protein testis specific | Transcription factor | 2 |
| BRD7 | BRD-containing 7 | Transcriptional repressor | 1 |
| BRD8A/B | BRD-containing 8A/B | TRRAP/TIP60 complex | 2 |
| BRD9 | BRD-containing 9 | Unknown | 1 |
| BRPF1A/B | Peregrin | MOZ complex subunit | 1 |
| BRPF3A | BRD and PHD-finger-containing | Unknown | 1 |
| | protein 3 | | |
| BRWD3 | BRD and WD-repeat-containing | JAK/STAT signalling | 2 |
| | protein 3 | | |
| CECR2 | Cat eye syndrome critical region 2 | Chromatin remodelling | 1 |
| CREBBP | CREB-binding protein | HAT | 1 |
| EP300 | HAT p300 | HAT | 1 |
| FALZ | Fetal Alzheimer antigene | Chromatin remodelling | 1 |
| GCN5L2 | General control of amino acid | HAT | 1 |
| | synthesis protein 5-like 2 | | |
| MLL | Mixed lineage leukaemia | Histone methyltransferase | 1 |
| PB1 | Polybromo | SWI/SNF PBAF subunit | 6 |
| PCAF | P300/CBP-associated factor | HAT | 1 |
| PHIP | PH-interacting protein | Insulin signalling | 2 |
| PRKCBP1 | Protein kinase C-binding protein 1 | Transcriptional regulator | 1 |
| SMARCA2A/B | SWI/SNF-related, matrix-associated, | SWI/SNF ATPase | 1 |
| | actin-dependent regulator of chromatin | | |
| SMARCA4 | SWI/SNF-related, matrix-associated, | SWI/SNF ATPase | 1 |
| | actin-dependent regulator of chromatin | | |
| SP100/SP110/SP140 | Nuclear body protein | Transcriptional regulator | |
| TAF1/TAF1L | Transcription initiation TFIID- | Transcription initiation | 2 |
| | associated factor | | |
| TRIM24/TRIM28/ | Transcription intermediary factor | Transcriptional silencer | 1 |
| TRIM33/TRIM66 | | | |
| WDR9 | BRD and WD-repeat-containing | Chromatin remodelling | 2 |
| | protein 1 | | |
| ZMYND11 | Zinc finger MYND-domain-containing | Corepressor | 1 |
| | protein 11 | | |

BRD, bromodomain; HAT, histone acetyltransferase; MOZ, monocytic leukaemia zinc finger protein; PHD, plant homology domain; SNF, sucrose nonfermenting.

In some embodiments, the chemotherapeutic agent is a composition comprising nanoparticles comprising a thiocolchicine derivative and a carrier protein (such as albumin).

In further embodiments a combination of therapeutic treatment agents is administered to breast cancer cells. The therapeutic agents may be administered serially (within minutes, hours, or days of each other) or in parallel; they also may be administered to the patient in a pre-mixed single composition. Combinations of breast cancer therapeutics include, but are not limited to the following: AT (Adriamycin and Taxotere), AC±T: (Adriamycin and Cytoxan, with or without Taxol or Taxotere), CMF (Cytoxan, methotrexate, and fluorouracil), CEF (Cytoxan, Ellence, and fluorouracil), FAC (fluorouracil, Adriamycin, and Cytoxan), CAF (Cytoxan, Adriamycin, and fluorouracil) (the FAC and CAF regimens use the same medicines but use different doses and frequencies), TAC (Taxotere, Adriamycin, and Cytoxan), and GET (Gemzar, Ellence, and Taxol).

Various combinations of more than an anticancer modality, agent or compound (or a combination of such agents and/or compounds) may be employed, for example, a first anticancer modality, agent or compound is “A” and a second anticancer modality, agent or compound (or a combination of such modalities, agents and/or compounds) given as part of an anticancer therapy regime, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic compounds or agents to a patient will follow general protocols for the administration of such compounds, taking into account the toxicity, if any, of the therapy. It is expected that the treatment cycles would be repeated as necessary. It also is contemplated that various standard therapies, as well as surgical intervention, may be applied in combination with the described therapy.

The term “a serine/threonine kinase inhibitor”, as used herein, relates to a compound which inhibits serine/threonine kinases. An example of a target of a serine/threonine kinase inhibitor includes, but is not limited to, dsRNA-dependent protein kinase (PKR). Examples of indirect targets of a serine/threonine kinase inhibitor include, but are not limited to, MCP-1, NF-kappaB, eIF2alpha, COX2, RANTES, IL8, CYP2A5, IGF-1, CYP2B1, CYP2B2, CYP2H1, ALAS-1, HIF-1, erythropoietin and/or CYP1A1. An example of a serine/theronin kinase inhibitor includes, but is not limited to, Sorafenib and 2-aminopurine, also known as 1H-purin-2-amine(9CI). Sorafenib is marketed as NEXAVAR.

The term “an angiogenesis inhibitor”, as used herein, relates to a compound which targets, decreases or inhibits the production of new blood vessels. Targets of an angiogenesis inhibitor include, but are not limited to, methionine aminopeptidase-2 (MetAP-2), macrophage inflammatory protein-1 (MIP-1a), CCL5, TGF-β, lipoxygenase, cyclooxygenase, and topoisomerase. Indirect targets of an angiogenesis inhibitor include, but are not limited to, p21, p53, CDK2 and collagen synthesis. Examples of an angiogenesis inhibitor include, but are not limited to, Fumagillin, which is known as 2,4,6,8-decatetraenedioic acid, mono[3R,4S,5S,6R)-5-methoxy-4-[(2R,3R)-2-methyl-3-(3-methyl-2-butenyl)oxi-ranyl]-1-oxaspiro[2.5]oct-6-yl]ester, (2E,4E,6E,8E)-(9CI); Shikonin, which is also known as 1,4-naphthalenedione, 5,8-dihydroxy-2-[(1R)-1-hydroxy-4-methyl-3-pentenyl]-(9CI); Tranilast, which is also known as benzoic acid, 2-[[3-(3,4-dimethoxyphenyl)-1-oxo-2-propenyl]amino]-(9CI); ursolic acid; suramin; thalidomide and lenalidomide, and marketed as REVLIMID.

Radiation therapy that cause DNA damage and have been used extensively include what are commonly known as □-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Approximately 60% of persons with cancer will undergo surgery of some type, which includes preventative, diagnostic or staging, curative and palliative surgery. Curative surgery is a cancer treatment that may be used in conjunction with other therapies, such as the treatment, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the treatment methods described herein may be used in conjunction with removal of superficial cancers, precancers, or incidental amounts of normal tissue.

Laser therapy is the use of high-intensity light to destroy tumor cells. Laser therapy affects the cells only in the treated area. Laser therapy may be used to destroy cancerous tissue and relieve a blockage in the esophagus when the cancer cannot be removed by surgery. The relief of a blockage can help to reduce symptoms, especially swallowing problems.

Photodynamic therapy (PDT), a type of laser therapy, involves the use of drugs that are absorbed by cancer cells; when exposed to a special light, the drugs become active and destroy the cancer cells. PDT may be used to relieve symptoms of esophageal cancer such as difficulty swallowing.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well. A patient may be administered a single compound or a combination of compounds described herein in an amount that is, is at least, or is at most 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg (or any range derivable therein). A patient may be administered a single compound or a combination of compounds described herein in an amount that is, is at least, or is at most 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500 mg/kg/day (or any range derivable therein).

Alternative cancer therapy include any cancer therapy other than surgery, chemotherapy and radiation therapy, such as immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

For example, the alternative cancer therapy may be a targeted therapy. The targeted therapy may be a RKIP-targeted treatment. In one embodiment of the method, the RKIP-targeted treatment used is a RKIP protein or expression vector or any agents that inbits downstream targets (e.g., Let-7 target genes, BACH1, HMGA1, MMP1, CXCR4, OPN) repressed by RKIP activity, such as antibodies that bind to any of these downstream targets. In a further embodiment, the inhibitory antibody is an intact antibody, i.e. a full-length antibody, or a fragment.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Gene therapy is the insertion of polynucleotides, including DNA or RNA, into an individual's cells and tissues to treat a disease. Antisense therapy is also a form of gene therapy. A therapeutic polynucleotide may be administered before, after, or at the same time of a first cancer therapy. Delivery of a vector encoding a variety of proteins is encompassed in certain aspects. For example, cellular expression of the exogenous tumor suppressor oncogenes would exert their function to inhibit excessive cellular proliferation, such as p53, p16 and C-CAM.

Additional agents to be used to improve the therapeutic efficacy of treatment include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with treatment methods described herein to improve the anti-hyperproliferative efficacy of the treatments. Inhibitors of cell adhesion are contemplated to improve the efficacy. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with treatment methods described herein to improve the treatment efficacy.

Hormonal therapy may also be used or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### IV. Pharmaceutical Compositions

In certain aspects, the compositions or agents for use in the methods, such as therapeutic agents or protein inhibitors, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active agent, such as an isolated exosome, a related lipid nanovesicle, or an exosome or nanovesicle loaded with therapeutic agents or diagnostic agents. In other embodiments, the active agent may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 microgram/kg/body weight to about 100 mg/kg/body weight, about 5 microgram/kg/body weight to about 500 milligram/kg/body weight, etc., can be administered.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### V. Kits

Certain aspects of the present disclosure also concern kits containing compositions of the disclosure or compositions to implement methods of the disclosure. In some embodiments, kits can be used to evaluate one or more nucleic acid and/or polypeptide molecules. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more nucleic acid probes, synthetic RNA molecules or inhibitors, or any value or range and combination derivable therein. In some embodiments, there are kits for evaluating gene expression, protein expression, or protein activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, polypeptide detecting agents, and/or inhibitors or antagonists of the disclosure for prognostic or diagnostic applications are included. Specifically contemplated are any such molecules corresponding to any nucleic acid or polypeptide identified herein.

In certain aspects, negative and/or positive control agents are included in some kit embodiments. The control molecules can be used to verify transfection efficiency and/or control for transfection-induced changes in cells.

Embodiments of the disclosure include kits for analysis of a pathological sample by assessing a nucleic acid or polypeptide profile for a sample comprising, in suitable container means, two or more RNA probes, or a polypeptide detecting agent, wherein the RNA probes or polypeptide detecting agent detects nucleic acids or polypeptides described herein. Furthermore, the probes, detecting agents and/or inhibiting reagents may be labeled. Labels are known in the art and also described herein. In some embodiments, the kit can further comprise reagents for labeling probes, nucleic acids, and/or detecting agents. The kit may also include labeling reagents, including at least one of amine-modified nucleotide, poly(A) polymerase, and poly(A) polymerase buffer. Labeling reagents can include an amine-reactive dye. Certain aspects also encompass kits for performing the diagnostic or therapeutic methods. Such kits can be prepared from readily available materials and reagents. For example, such kits can comprise any one or more of the following materials: enzymes, reaction tubes, buffers, detergent, primers, probes, antibodies. In a particular embodiment, these kits allow a practitioner to obtain samples of neoplastic cells in breast, blood, tears, semen, saliva, urine, tissue, serum, stool, sputum, cerebrospinal fluid and supernatant from cell lysate. In another particular embodiment, these kits include the needed apparatus for performing RNA extraction, RT-PCR, and gel electrophoresis. Instructions for performing the assays can also be included in the kits.

In a particular aspect, these kits may comprise a plurality of agents for assessing the differential expression of a plurality of biomarkers, wherein the kit is housed in a container. The kits may further comprise instructions for using the kit for assessing expression, means for converting the expression data into expression values and/or means for analyzing the expression values to generate prognosis. The agents in the kit for measuring biomarker expression may comprise a plurality of PCR probes and/or primers for qRT-PCR and/or a plurality of antibody or fragments thereof for assessing expression of the biomarkers. In another embodiment, the agents in the kit for measuring biomarker expression may comprise an array of polynucleotides complementary to the mRNAs of the biomarkers. Possible means for converting the expression data into expression values and for analyzing the expression values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Metastasis Suppressors Regulate the Tumor Microenvironment by Blocking Recruitment of Pro-Metastatic Tumor-Associated Macrophages

Triple-negative breast cancer (TNBC) patients have the highest risk of recurrence and metastasis. Because some cannot be treated with targeted therapies, and may not respond to chemotherapy, they represent a clinically underserved group. TNBC is characterized by reduced expression of metastasis suppressors such as Raf Kinase Inhibitory Protein (RKIP), which inhibits tumor invasiveness. Mechanisms by which metastasis suppressors alter tumor cells are well characterized; however, their ability to regulate the tumor microenvironment, and the importance of such regulation to metastasis suppression is incompletely understood.

Here, Applicants use a species-specific RNAseq to show that RKIP expression in tumors markedly reduces the number and metastatic potential of infiltrating TAMs. TAMs isolated from non-metastatic RKIP+ tumors, relative to metastatic RKIP-tumors, exhibit a reduced ability to drive tumor cell invasion and decreased secretion of pro-metastatic factors including GZMB, VEGFD, MMP12, VEGFA, TNFR2, and GRN. RKIP regulates TAM recruitment by blocking HMGA2, resulting in reduced expression of numerous macrophage chemotactic factors, including CCL5. Interestingly, CCL5 overexpression in RKIP+ tumors restores recruitment of pro-metastatic TAMs and intravasation, highlighting the importance of RKIP-TAM interactions in regulating tumor invasiveness. The clinical significance of these interactions is underscored by Applicant's demonstration that a signature comprised of RKIP signaling and pro-metastatic TAM factors strikingly separates TNBC patients based on outcome. Collectively, the findings described in this example identify TAMs as a previously unsuspected mechanism by which the metastasis suppressor RKIP regulates tumor invasiveness, and further suggest that TNBC patients with decreased RKIP activity and increased TAM infiltration may respond to macrophage-based therapeutics.

Of the approximately 40,000 women diagnosed with breast cancer each year, 15-20% will have triple-negative breast cancer (TNBC). The most aggressive subset of breast cancer, TNBCs lack expression of the estrogen, progesterone and HER2/neu receptors. While clinical outcomes have improved for many patients with breast cancer, TNBC patients have higher rates of metastasis and more aggressive tumors, leading to heavy disease burden and early recurrence (Howlader N, Noone A, Krapcho M, Garshell J, Miller D, Altekruse S, et al. Cancer Statistics Review, 1975-2011-SEER Satistics. SEER Cancer Statistics Review 2011). Additionally, this disease disproportionately affects African-American women and women with lower incomes, with rates approximately three times higher in African-American women compared to the rest of the population (Carey L A, Perou C M, Livasy C A, Dressler L G, Cowan D, Conway K, et al. Race, breast cancer subtypes, and survival in the Carolina Breast Cancer Study. JAMA. 2006; 295:2492-502 and Anders C K, Carey L A. Biology, metastatic patterns, and treatment of patients with triplenegative breast cancer. Clin Breast Cancer. 2009; 9 Suppl 2:S73-81). Currently, the only form of therapy for these patients is surgery and platinum based chemotherapy; however, fewer than 30% of patients are responsive (Foulkes W D, Smith I E, Reis-Filho J S. Triple-negative breast cancer. N Engl J Med. 2010; 363:1938-48). Therefore, there is interest in novel approaches for treating TNBC patients, including targeting of the tumor stroma (Tchou J, Conejo-Garcia J. Targeting the tumor stroma as a novel treatment strategy for breast cancer: shifting from the neoplastic cell-centric to a stroma-centric paradigm. Advances in pharmacology. 2012; 65:45-61).

One possible strategy is to mimic the action of physiological tumor metastasis suppressors such as Raf Kinase Inhibitory Protein (RKIP). RKIP, a member of the evolutionarily conserved phosphatidylethanolamine family, has been implicated as a metastasis suppressor for prostate, breast and other solid tumors (Zeng L, Imamoto A, Rosner M R. Raf kinase inhibitory protein (RKIP): a physiological regulator and future therapeutic target. Expert Opin Ther Targets. 2008; 12:1275-87; Fu Z, Smith P C, Zhang L, Rubin M A, Dunn R L, Yao Z, et al. Effects of raf kinase inhibitor protein expression on suppression of prostate cancer metastasis. J Natl Cancer Inst. 2003; 95:878-89; and Dangi-Garimella S, Yun J, Eves E M, Newman M, Erkeland S J, Hammond S M, et al. Raf kinase inhibitory protein suppresses a metastasis signalling cascade involving LIN28 and let-7. EMBO J. 2009; 28:347-58). RKIP inhibits key signaling pathways including Raf/MAP kinase, GRK2-regulated ß-adrenergic receptor, and NFkB activation (Zeng L, Imamoto A, Rosner M R. Raf kinase inhibitory protein (RKIP): a physiological regulator and future therapeutic target. Expert Opin Ther Targets. 2008; 12:1275-87). Previously, RKIP was shown to suppress the expression of many pro-metastatic genes in TNBC cells by inhibiting transcriptional regulators such as HMGA2 (Dangi-Garimella S, Yun J, Eves E M, Newman M, Erkeland S J, Hammond S M, et al. Raf kinase inhibitory protein suppresses a metastasis signalling cascade involving LIN28 and let-7. EMBO J. 2009; 28:347-58; Yun J, Frankenberger C A, Kuo W L, Boelens M C, Eves E M, Cheng N, et al. Signalling pathway for RKIP and Let-7 regulates and predicts metastatic breast cancer. EMBO J. 2011; 30:4500-14; and Yeung K C, Rose D W, Dhillon A S, Yaros D, Gustafsson M, Chatterjee D, et al. Raf kinase inhibitor protein interacts with NF-kappaB-inducing kinase and TAK1 and inhibits NFkappaB activation. Mol Cell Biol. 2001; 21:7207-17). Because previous studies have focused on the effects of metastasis suppressors in tumor cells, their role in regulating the tumor microenvironment is unknown.

Recent evidence has shown that the microenvironment regulates both tumor progression and metastasis. In particular, macrophages have been shown to play a dual role in tumor growth, either driving tumor rejection or tumor progression depending on the type of macrophage activation (Wynn T A, Chawla A, Pollard J W. Macrophage biology in development, homeostasis and disease. Nature. 2013; 496:445-55). Classical activation of macrophages by IFNγ, lipopolysaccharide (LPS), or tumor necrosis factor-α (TNFα) leads to polarization of M1 macrophages that secrete inflammatory cytokines important in the body's anti-tumor response. M2 macrophages, activated by factors such as IL4, play an essential role in wound healing. Secretion of factors from tumor-associated macrophages (TAMs), thought to be M2, leads to tumor growth, progression, and metastasis (Agarwal S, Gertler F B, Balsamo M, Condeelis J S, Camp R L, Xue X, et al. Quantitative assessment of invasive mena isoforms (Menacalc) as an independent prognostic marker in breast cancer. Breast Cancer Res. 2012; 14:R124; Joyce J A, Pollard J W. Microenvironmental regulation of metastasis. Nat Rev Cancer. 2009; 9:239-52; and Green C E, Liu T, Montel V, Hsiao G, Lester R D, Subramaniam S, et al. Chemoattractant signaling between tumor cells and macrophages regulates cancer cell migration, metastasis and neovascularization. PLoS One. 2009; 4:e6713) as well drug resistance (De Palma M, Lewis C E. Macrophage regulation of tumor responses to anticancer therapies. Cancer Cell. 2013; 23:277-86). However, recent evidence suggests that this division of macrophages into two discrete subtypes incompletely describes the range of macrophage phenotypes present in the tumor microenvironment (Meissner F, Scheltema R A, Mollenkopf H J, Mann M. Direct proteomic quantification of the secretome of activated immune cells. Science. 2013; 340:475-8). Importantly, studies of breast cancer patients show that CD163+ macrophage recruitment positively correlates with TNBC while negatively correlating with ER+ and luminal tumors (Medrek C, Ponten F, Jirstrom K, Leandersson K. The presence of tumor associated macrophages in tumor stroma as a prognostic marker for breast cancer patients. BMC Cancer. 2012; 12:306). Therefore, recruitment of alternatively activated TAMs could play a significant role in the outcome of TNBC patients and explain their poor prognosis.

TAMs are recruited to mammary tumors through induction of a variety of cytokines and chemokines, where they play essential roles in driving metastasis. For example, TAMs recruited by CSF-1 show higher levels of VEGF-A, with increased angiogenesis in the polyoma middle T genetically engineered mouse model for breast cancer (Lin E Y, Li J F, Gnatovskiy L, Deng Y, Zhu L, Grzesik D A, et al. Macrophages regulate the angiogenic switch in a mouse model of breast cancer. Cancer Res. 2006; 66:11238-46). Similarly, CCL2 was required for TAM infiltration in primary breast tumors as well as TAM-enabled metastatic colonization of lungs (Qian B Z, Li J, Zhang H, Kitamura T, Zhang J, Campion L R, et al. CCL2 recruits inflammatory monocytes to facilitate breast-tumour metastasis. Nature. 2011; 475:222-5). Antagonists of CCL5 inhibited TAM recruitment in a syngeneic mouse model (Robinson S C, Scott K A, Wilson J L, Thompson R G, Proudfoot A E, Balkwill F R. A chemokine receptor antagonist inhibits experimental breast tumor growth. Cancer Res. 2003; 63:8360-5). Finally, recent work comparing breast tumors before and after EMT has shown that GM-CSF is able to recruit TAMs to the primary tumor (Lu H, Clauser K R, Tam W L, Frose J, Ye X, Eaton E N, et al. A breast cancer stem cell niche supported by juxtacrine signalling from monocytes and macrophages. Nat Cell Biol. 2014; 16:1105-17; and Su S, Liu Q, Chen J, Chen J, Chen F, He C, et al. A positive feedback loop between mesenchymal-like cancer cells and macrophages is essential to breast cancer metastasis. Cancer Cell. 2014; 25:605-20). Although factors enabling recruitment of prometastatic TAMs to mammary tumors have been identified, the regulation of these pathways by metastasis suppressors and the specific phenotypes of these TAMs is poorly understood.

In this example, Applicants combine species-specific RNA sequencing, protein secretion profiling, functional assays, and gene knockout studies in xenograft and syngeneic breast cancer models to characterize the effects of the metastasis suppressor RKIP on TAMs and to identify the molecular mechanisms that mediate these effects. These findings demonstrate that RKIP blocks a subset of TAMs that secrete pro-metastatic factors and is enriched in human TNBC patients, suggesting that one mechanism by which metastasis suppressors alter tumor invasiveness is by regulating TAMs.

### A. Results

1. Non-Metastatic RKIP+ Tumors Contain Fewer Macrophages

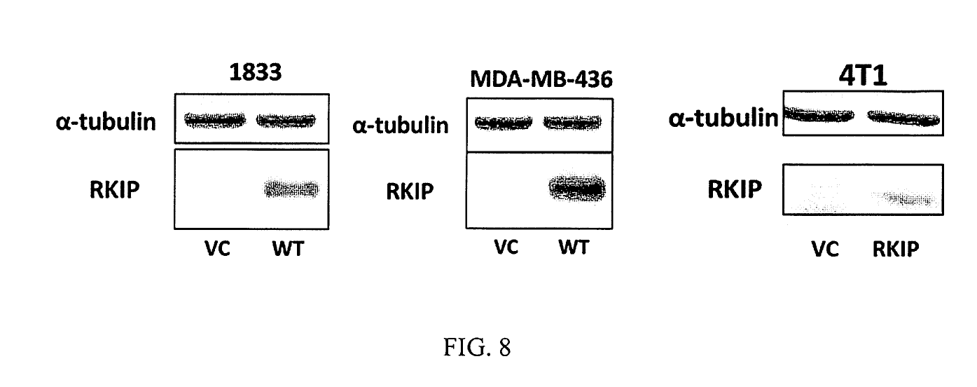

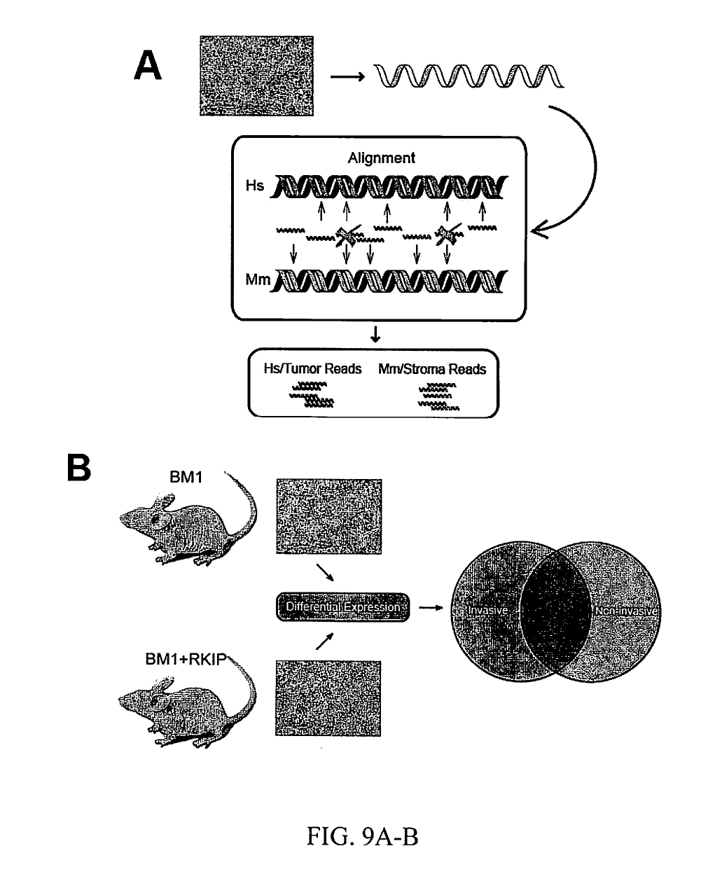

To compare metastatic and non-metastatic tumors that were otherwise isogenic, Applicants used highly metastatic BM1 derivatives of the MDA-MB-231 human TNBC cell line that stably express either the metastasis suppressor Raf Kinase Inhibitory Protein (RKIP+) or a vector control (control) (FIG. 8) (Dangi-Garimella S, Yun J, Eves E M, Newman M, Erkeland S J, Hammond S M, et al. Raf kinase inhibitory protein suppresses a metastasis signalling cascade involving LIN28 and let-7. EMBO J. 2009; 28:347-58 and Yun J, Frankenberger C A, Kuo W L, Boelens M C, Eves E M, Cheng N, et al. Signalling pathway for RKIP and Let-7 regulates and predicts metastatic breast cancer. EMBO J. 2011; 30:4500-14). Tumor cells were injected orthopically into athymic nude mice, and RNA from the tumors was then isolated and sequenced. To overcome the challenge of distinguishing between tumor-specific and stroma-specific gene expression, Applicants used nextgeneration RNA sequencing (RNAseq) in this xenograft mouse model to separate sequencing reads based on their species of origin (FIG. 9).

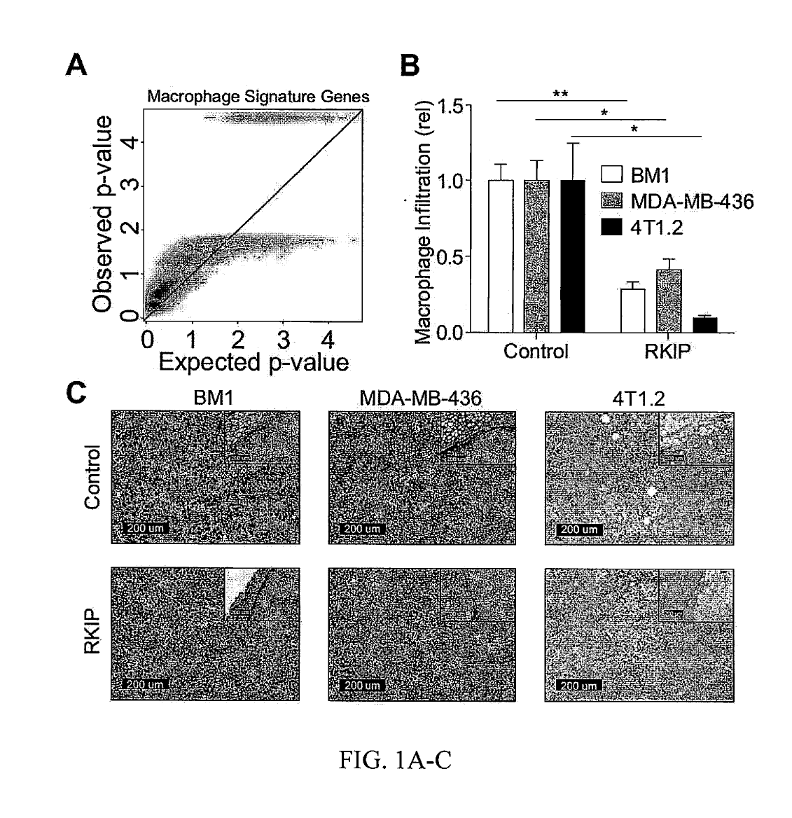

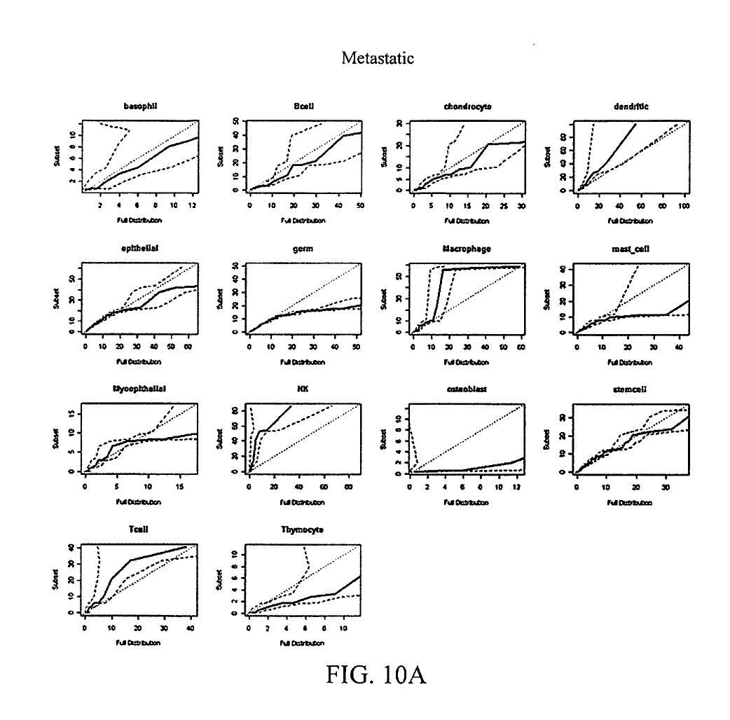

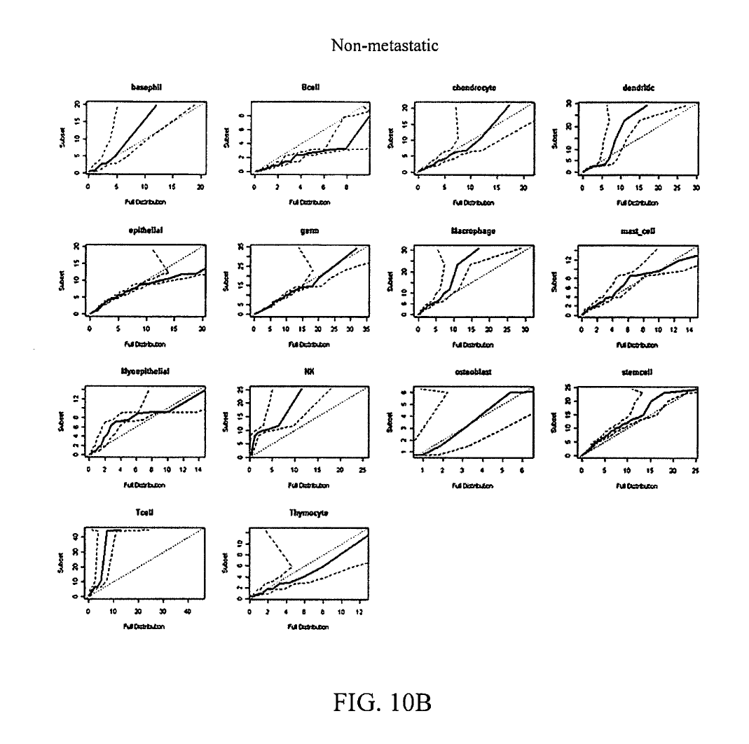

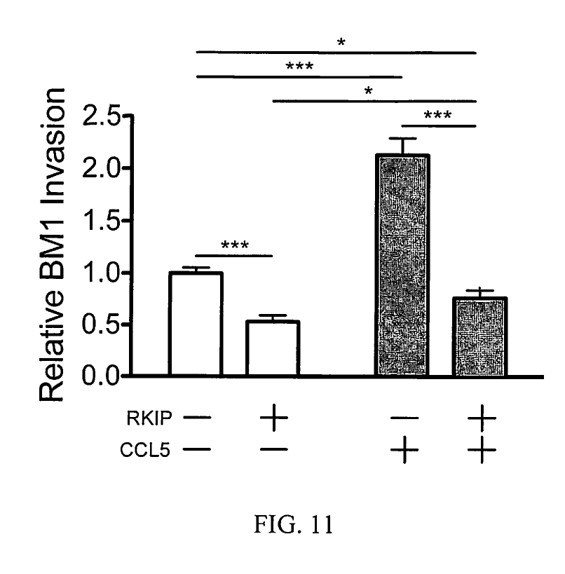

Analysis of gene expression changes between RKIP+ and control tumor cells using GOseq revealed that the most significant difference was the immune response and, specifically, macrophage chemotaxis (Q-value=2.2×10−4, Table 1). Using mouse gene sets (Table 1) characteristic of common immune cell types (Hijikata A, Kitamura H, Kimura Y, Yokoyama R, Aiba Y, Bao Y, et al. Construction of an open-access database that integrates cross-reference information from the transcriptome and proteome of immune cells. Bioinformatics. 2007; 23:2934-41), Applicants observed a clear depletion of gene expression associated with macrophages in the RKIP+ tumor microenvironment (FIG. 1A), and this was the most robust change observed (FIG. 10). Immunohistochemical staining confirmed a marked reduction in the number of tumor-associated macrophages (TAMs) at the primary tumor site in RKIP+ tumors relative to controls, both in xenograft (BM1, MDA-MB-436) and syngeneic (4T1.2) tumor models (FIG. 1B-C; 15).

TABLE 1
Ontological categories enriched among genes whose expression
level is significantly decreased by RKIP relative in TNBC
xenograft tumors. (FDR = 0.01 for all comparisons).

| GOid | Category | Qvalue |
|---|---|---|
| GO:0032103 | positive regulation of response to | 1.3E−05 |
| | external stimulus | |
| GO:0006953 | acute-phase response | 1.55E−05 |
| GO:0010759 | positive regulation of macrophage | 2.98E−05 |
| | chemotaxis | |
| GO:0010758 | regulation of macrophage chemotaxis | 3.94E−05 |
| GO:0006955 | immune response | 4.1E−05 |
| GO:0071622 | regulation of granulocyte chemotaxis | 4.6E−05 |
| GO:0050921 | positive regulation of chemotaxis | 4.91E−05 |
| GO:0048520 | positive regulation of behavior | 5.5E−05 |
| GO:0071675 | regulation of mononuclear cell migration | 5.71E−05 |
| GO:0030169 | low-density lipoprotein particle binding | 7.67E−05 |
| GO:0071674 | mononuclear cell migration | 7.69E−05 |
| GO:0071621 | granulocyte chemotaxis | 8.47E−05 |
| GO:0071345 | cellular response to cytokine stimulus | 0.000113246 |
| GO:0050920 | regulation of chemotaxis | 0.000124252 |
| GO:0002526 | acute inflammatory response | 0.000131065 |
| GO:0071813 | lipoprotein particle binding | 0.00021975 |
| GO:0071814 | protein-lipid complex binding | 0.00021975 |
| GO:0048246 | macrophage chemotaxis | 0.00022022 |
| GO:0050795 | regulation of behavior | 0.000222061 |
| GO:0045087 | innate immune response | 0.000225704 |
| GO:0070098 | chemokine-mediated signaling pathway | 0.000230038 |
| GO:0034097 | response to cytokine stimulus | 0.000294786 |
| GO:0030246 | carbohydrate binding | 0.000440636 |

2. RKIP Suppresses Recruitment of a Distinct TAM Population that Potentiates Tumor Cell Invasion

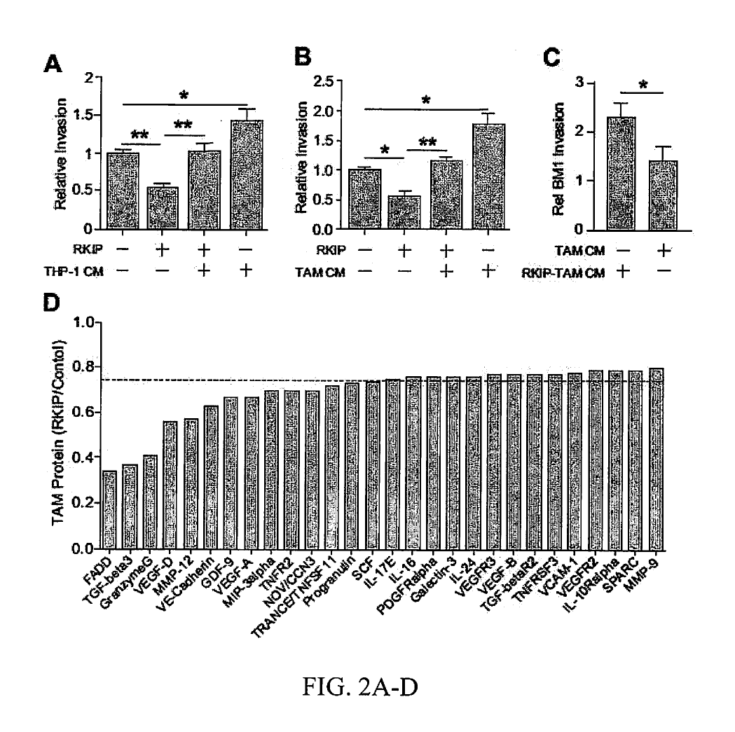

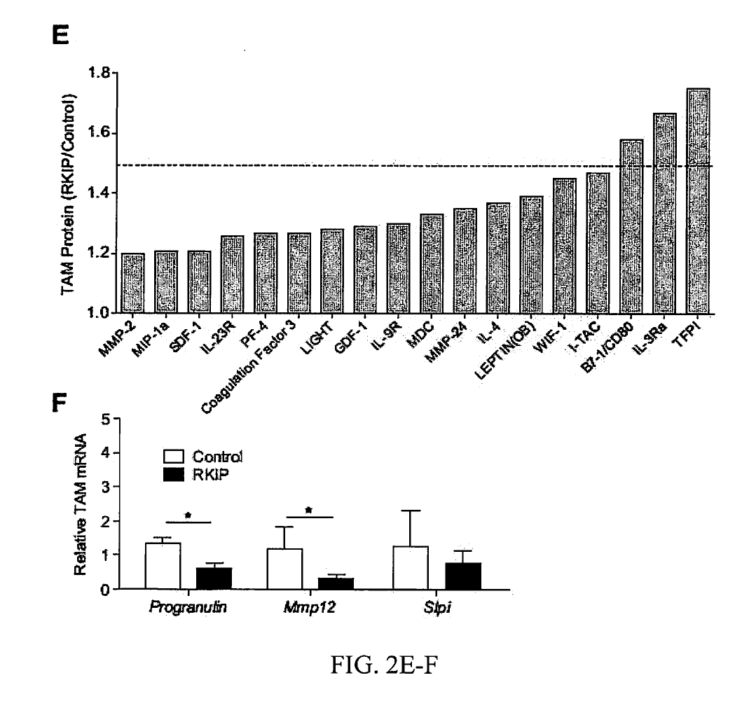

Since RKIP regulates the number of macrophages in tumors (FIG. 1A-C) and TAMs are known to play a significant role in tumor biology (Wynn T A, Chawla A, Pollard J W. Macrophage biology in development, homeostasis and disease. Nature. 2013; 496:445-55), Applicants hypothesized that changes in TAMs may in part explain suppression of intravasation by RKIP. In support of this hypothesis, treating RKIP+ BM1 tumor cells with conditioned media (CM) from THP1 cells, a TAM-like human monocyte cell line, could restore tumor cell invasion relative to levels observed in control BM1 tumor cells (FIG. 2A). Similar results were observed with the CM of TAMs purified from control BM1 tumors (FIG. 2B). These findings demonstrate that TAMs from metastatic tumors can overcome blockade of tumor cell invasion by RKIP.

It is well established that, depending on environmental conditions, TAMs can adopt phenotypes with pro-tumor (“M2-like”) or anti-tumor (“M1-like”) properties (13). Applicants therefore explored the possibility that, in addition to reducing the number of macrophages in tumors, RKIP might also alter their functional properties to suppress metastasis. To test this hypothesis, Applicants assessed the functional phenotype of TAMs purified from BM1 tumors (metastatic) and RKIP+ BM1 tumors (non-metastatic) using two interrelated approaches.

Initially, Applicants quantified the effect of TAM conditioned media on tumor cell invasion in vitro. Pretreating BM1 tumor cells with the conditioned media (CM) of TAMs isolated from control BM1 tumors, like THP1 cells, potentiated invasion (FIG. 2B). In sharp contrast, factors secreted by TAMs from RKIP+ BM1 tumors had no significant effect on tumor cell invasiveness (FIG. 2C). These results indicate that the TAMs from metastatic and non-metastatic (RKIP+) tumors have distinct phenotypes.

Applicants then used a RayBiotech cytokine array to quantify the relative abundance of 400 genes including inflammatory and tumorigenic factors (eg. cytokines, growth factors, etc.) in the TAM conditioned media. TAMs purified from RKIP+ BM1 tumors relative to control BM1 tumors were distinguished by reduced abundance of a number of pro-metastatic factors (Wynn T A, Chawla A, Pollard J W. Macrophage biology in development, homeostasis and disease. Nature. 2013; 496:445-55) including TGF-β3, VEGF-D, MMP-12, GDF-9, VEGF, TNFR2, and GRN (FIG. 2D). Applicants also observed induction of secreted factors in the CM of RKIP+ BM1 tumors including CD80 and TFPI, two potential anti-tumor proteins (24-26). Finally, Applicants confirmed differential regulation of Mmp12 and Gm (progranulin) transcripts in TAMs isolated from BM1 versus RKIP+ BM1 tumors by qRTPCR (FIG. 2E). Taken together, the direct functional evidence and protein expression analysis suggest that RKIP suppresses recruitment of a TAM population that secretes a set of proinvasive and pro-metastatic proteins.

3. Overexpression of CCL5 Restores TAMs and Overcomes Metastasis Suppression in RKIP+ Tumors.

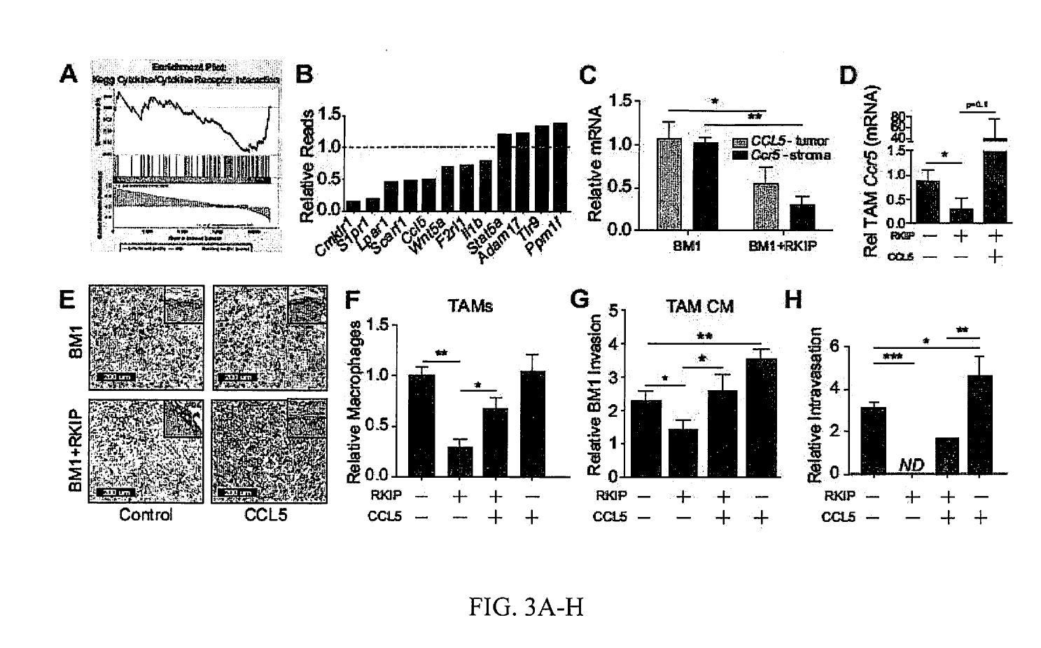

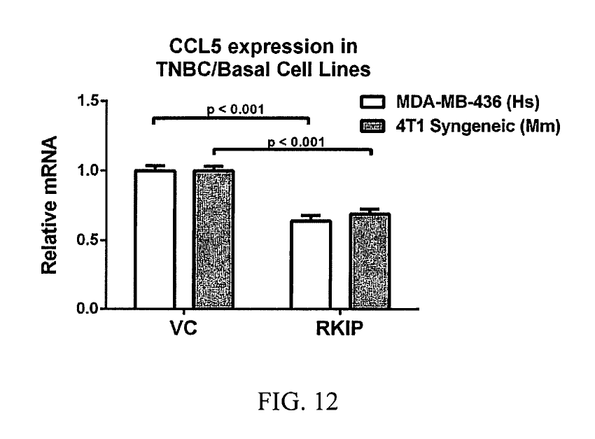

To determine the mechanism by which RKIP regulates TAM number and function, Applicants examined our RNAseq data comparing BM1 and RKIP+ BM1 tumors. RKIP+ tumors demonstrated a general suppression of numerous genes involved in cytokine/cytokine receptor interactions, particularly in relation to external stimulus and macrophage chemotaxis (FIG. 3A; Table 1) (FDR=3.9×10−2). The foremost of the chemokine factors was CCL5 (FIG. 3B) (Robinson S C, Scott K A, Wilson J L, Thompson R G, Proudfoot A E, Balkwill F R. A chemokine receptor antagonist inhibits experimental breast tumor growth. Cancer Res. 2003; 63:8360-5 and Karnoub A E, Dash A B, Vo A P, Sullivan A, Brooks M W, Bell G W, et al. Mesenchymal stem cells within tumour stroma promote breast cancer metastasis. Nature. 2007; 449:557-63). Applicants therefore analyzed CCL5 expression by species-specific qRT-PCR and confirmed its downregulation in RKIP+ tumors (FIG. 3C). Similar decreases in CCL5 transcripts were observed following RKIP expression in human MDA-MB-436 and mouse 4T1.2 tumor cell lines (FIG. 12). Thus, RKIP suppresses CCL5 expression in multiple human and murine tumor cell lines.

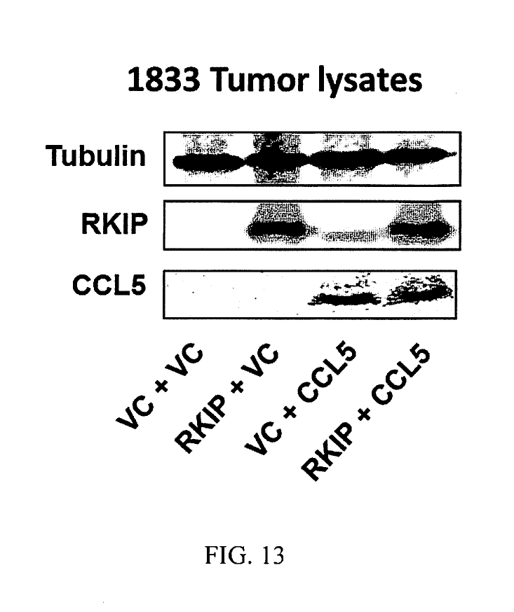

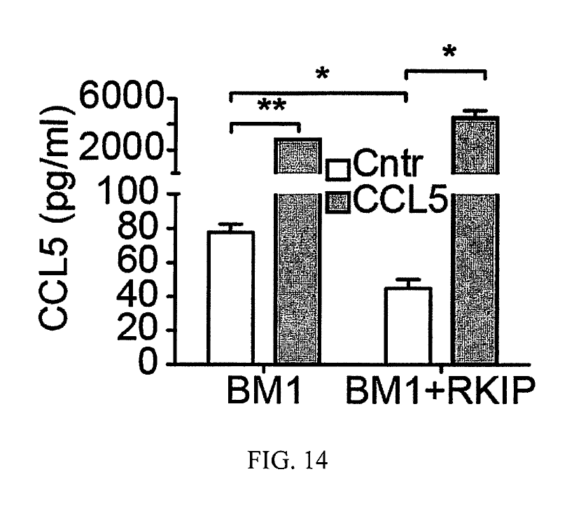

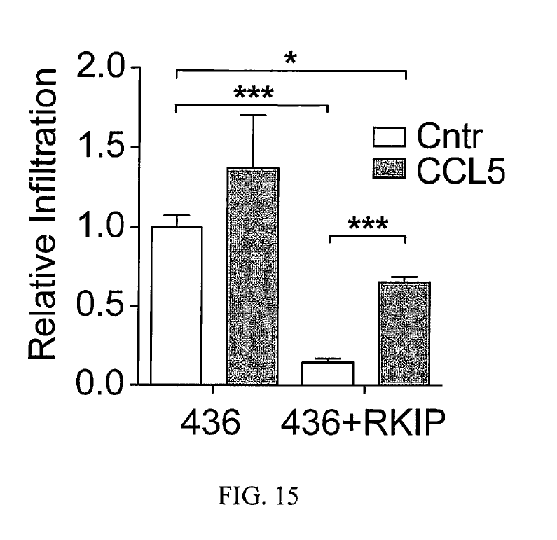

Applicants used two approaches to investigate whether suppression of CCL5 by RKIP plays an important role in regulating macrophage accumulation into tumors in vivo. First, since CCL5 recruits macrophages via interaction with its receptor CCR5 (Robinson S C, Scott K A, Wilson J L, Thompson R G, Proudfoot A E, Balkwill F R. A chemokine receptor antagonist inhibits experimental breast tumor growth. Cancer Res. 2003; 63:8360-5 and Velasco-Velazquez M, Jiao X, De La Fuente M, Pestell T G, Ertel A, Lisanti M P, et al. CCR5 antagonist blocks metastasis of basal breast cancer cells. Cancer Res. 2012; 72:3839-50), Applicants measured Ccr5 expression in the stroma. Applicants found that Ccr5 levels were significantly reduced in RKIP+ tumors (FIG. 3C). In particular, Applicants observed a significant decrease in Ccr5 transcript expression in BM1 RKIP+ TAMs relative to control (FIG. 3D). Second, we transfected CCL5 into BM1 and MDAMB-436 cells stably expressing RKIP or control vector (FIG. 13-14) and found an increase in the number of Ccr5+ macrophages in tumors expressing RKIP and CCL5 (RKIP+CCL5) compared to those just expressing RKIP alone (RKIP+) (FIGS. 3C,D,E). These findings suggest that modulation of CCL5 expression is one important mechanism by which RKIP controls tumor macrophage recruitment.

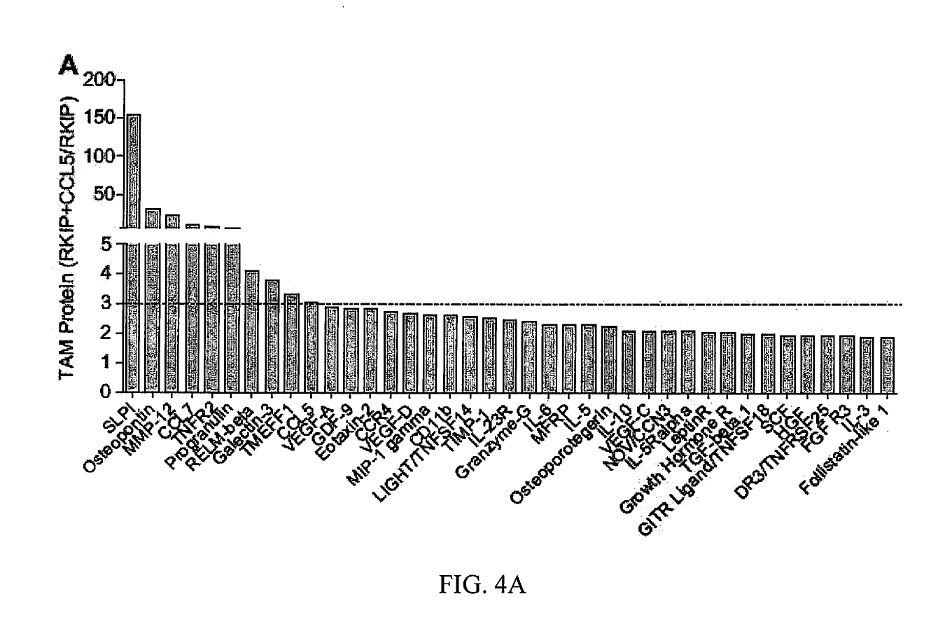

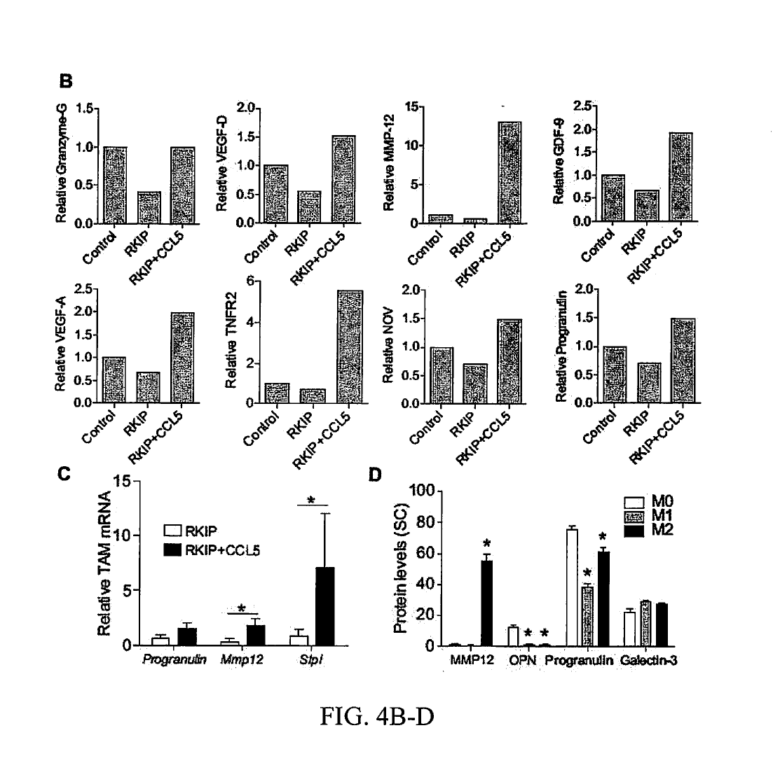

To determine if CCL5 overexpression in RKIP+ BM1 tumors could also restore a TAM phenotype that promotes tumor invasion, Applicants first conducted functional assays. Whereas TAMs isolated from BM1 RKIP+ tumors had no effect on BM1 invasion; TAMs isolated from RKIP+ BM1 tumors overexpressing CCL5 induced tumor cell invasion with similar efficiency as TAMs isolated from metastatic BM1 tumors (FIG. 4A). Since invasion enables tumor cell entry into vessels, Applicants then investigated whether overexpression of CCL5 in RKIP+ BM1 tumor cells (FIGS. 13-14) could overcome the inhibitory effect of RKIP on intravasation. Consistent with RKIP's ability to suppress metastasis, RKIP expression in BM1 tumor cells potently inhibited intravasation into blood vessels (FIG. 3F)(Yun J, Frankenberger C A, Kuo W L, Boelens M C, Eves E M, Cheng N, et al. Signalling pathway for RKIP and Let-7 regulates and predicts metastatic breast cancer. EMBO J. 2011; 30:4500-14). Importantly, elevating CCL5 expression in RKIP+ BM1 cells produced a partial but significant recovery of tumor cell intravasation into blood vessels (FIG. 3F). The CCL5 stimulation is not specific to RKIP, since overexpression of CCL5 in control metastatic tumor cells potentiated both invasion (FIG. 4A) and intravasation (FIG. 3F).

Applicants then determined whether CCL5 overexpression in BM1+RKIP tumors could enable recruitment of TAMs that secrete pro-metastatic factors. Analysis of proteins in the CM of isolated TAMs by cytokine arrays revealed robust induction of a number of factors that were suppressed in TAMs recruited to non-metastatic tumors (FIG. 4B). For example, GZMB (Granzyme-B), VEGF-D, MMP-12, GDF-9, VEGF-A, NOV, TNFR2, and GRN (Progranulin) were expressed by TAMs recruited to BM1 tumors, inhibited in TAMs recruited to BM1 RKIP+ tumors, and restored or even overexpressed in TAMs recruited to BM1 RKIP+CCL5 tumors (FIG. 4C). Other factors were also robustly induced by CCL5 including SLPI. Applicants confirmed the Induction of selected factors including MMP12, SLPI, and GRN in TAMs isolated from BM1 RKIP+CCL5 tumors by qRT-PCR (FIG. 4D). These results identify a select group of factors that are characteristic of TAMs recruited by CCL5 to metastatic tumors and suppressed in TAMs recruited to non-metastatic (RKIP+) tumors.

The difference between TAMs from metastatic and non-metastatic (RKIP+) tumors could reflect a switch from an M2 to an M1 phenotype. To examine this possibility, Applicants analyzed proteins secreted by bone marrow-derived macrophages (MO), M1 macrophages (activated by LPS/IFNγ), and M2 macrophages (activated by IL4) using mass spectrometry. When Applicants compared them to factors secreted by CCL5-recruited TAMs (FIG. 4C), MMP12 was significantly increased in M2 compared to MO and M1 macrophages; however, GRN and LGALS3 (Galectin-3) were broadly expressed, and osteopontin (OPN/SPP1) was selectively expressed in MO macrophages (FIG. 4E). These results suggest that the markers expressed in the CCL5-recruited TAMs (Tsukamoto A S, Grosschedl R, Guzman R C, Parslow T, Varmus H E. Expression of the int-1 gene in transgenic mice is associated with mammary gland hyperplasia and adenocarcinomas in male and female mice. Cell. 1988; 55:619-25) are not indicative of an M1 nor M2 phenotype.

Collectively, these findings demonstrate that CCL5 overexpression can promote macrophage infiltration, macrophage function, and intravasation on a non-metastatic (RKIP+) background, suggesting that downregulation of CCL5 by RKIP, and the concomitant reduction in TAMs, may be an important mechanism by which RKIP suppresses metastasis.

Suppression of metastasis and TAMs by RKIP is coordinated though HMGA2 signaling

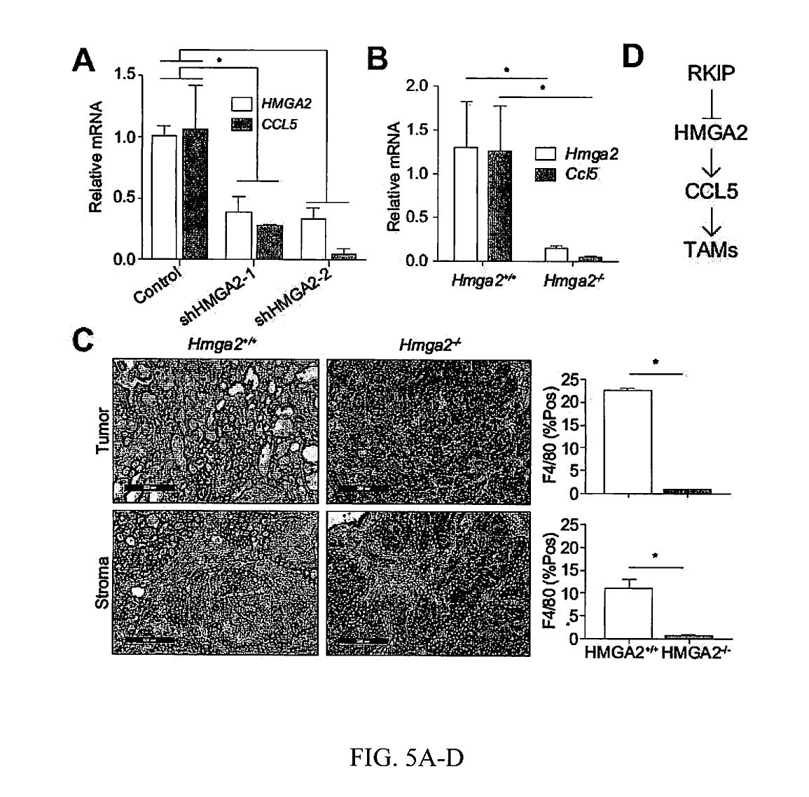

Applicant's previous work showed that RKIP suppresses breast cancer metastasis in part by inhibiting the architectural transcription factor High-mobility group AT-hook 2 (HMGA2) (Dangi-Garimella S, Yun J, Eves E M, Newman M, Erkeland S J, Hammond S M, et al. Raf kinase inhibitory protein suppresses a metastasis signalling cascade involving LIN28 and let-7. EMBO J. 2009; 28:347-58 and Yun J, Frankenberger C A, Kuo W L, Boelens M C, Eves E M, Cheng N, et al. Signalling pathway for RKIP and Let-7 regulates and predicts metastatic breast cancer. EMBO J. 2011; 30:4500-14). Applicants therefore determined whether RKIP suppresses macrophage recruitment via a similar mechanism. HMGA2 depletion in BM1 cells led to a significant decrease in CCL5 expression in vitro (FIG. 5A). To test whether HMGA2 regulates macrophage accumulation in vivo, Applicants crossed Hmga2−/− mice with the invasive MMTV-Wnt1 genetically engineered mouse (GEM; (Tsukamoto A S, Grosschedl R, Guzman R C, Parslow T, Varmus H E. Expression of the int-1 gene in transgenic mice is associated with mammary gland hyperplasia and adenocarcinomas in male and female mice. Cell. 1988; 55:619-25)). Similar to the RKIP-phenotype, Hmga2−/− GEM mice (relative to Hmga2+/+) had decreased Ccl5 expression in the mammary tumors (FIG. 5B) and a marked reduction in the number of macrophages present both in the tumor tissue as well as in the surrounding stroma (FIG. 5C). Together with previous findings (Dangi-Garimella S, Yun J, Eves E M, Newman M, Erkeland S J, Hammond S M, et al. Raf kinase inhibitory protein suppresses a metastasis signalling cascade involving LIN28 and let-7. EMBO J. 2009; 28:347-58 and Yun J, Frankenberger C A, Kuo W L, Boelens M C, Eves E M, Cheng N, et al. Signalling pathway for RKIP and Let-7 regulates and predicts metastatic breast cancer. EMBO J. 2011; 30:4500-14), these results suggest that RKIP suppression of metastasis, tumor cell CCL5 expression, and macrophage recruitment is coordinated through HMGA2 signaling (FIG. 5D).

4. An RKIP-Macrophage Gene Signature Predicts Metastasis-Free Survival

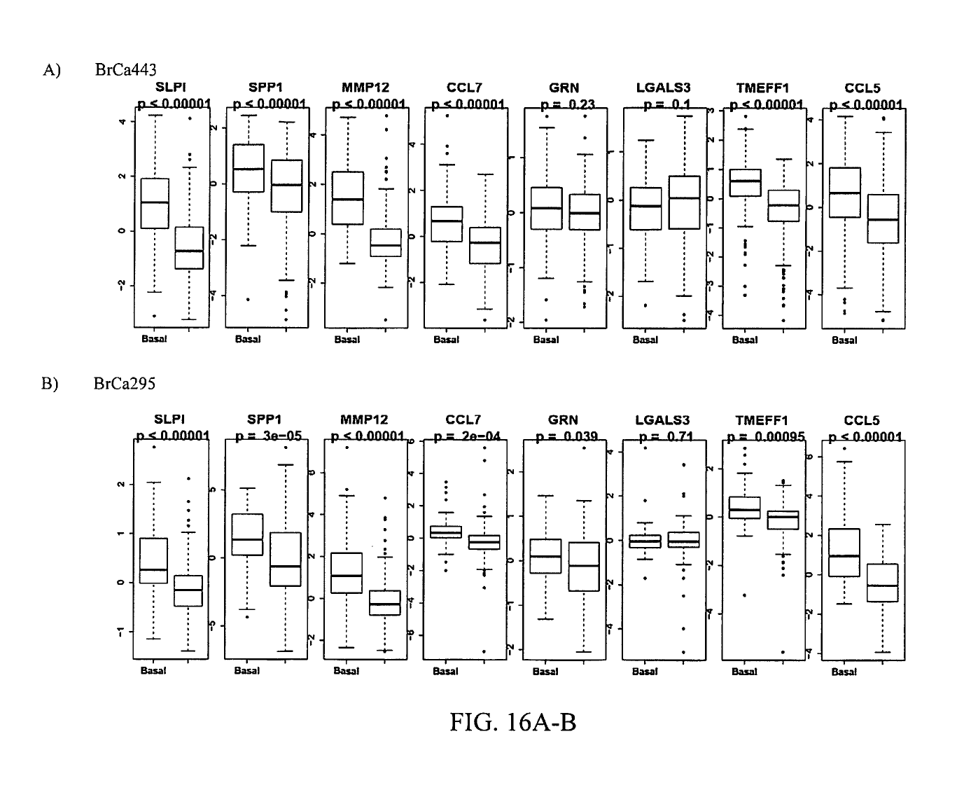

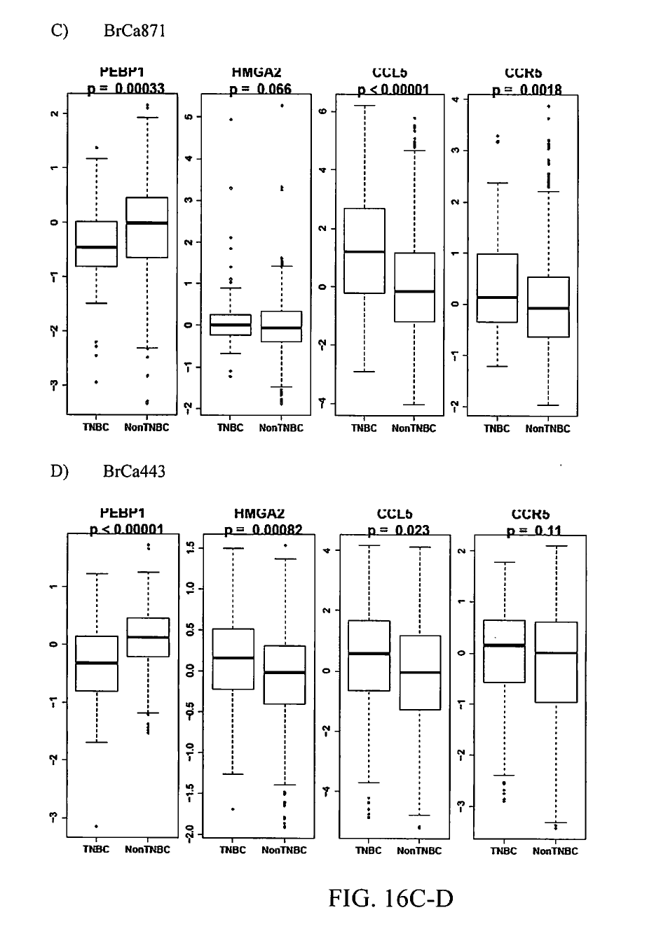

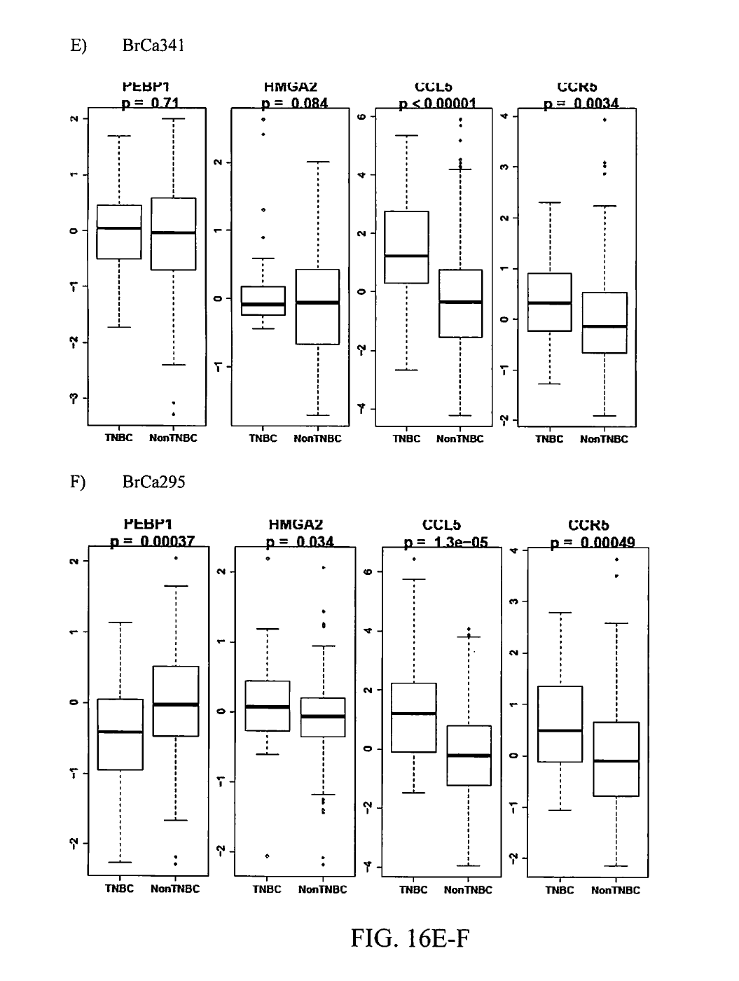

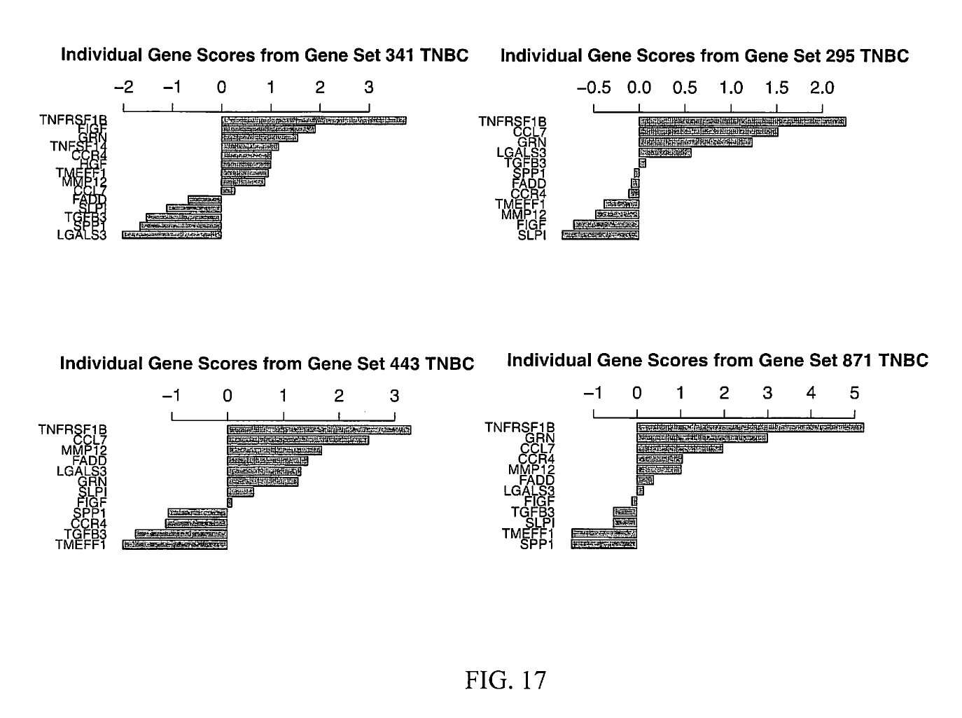

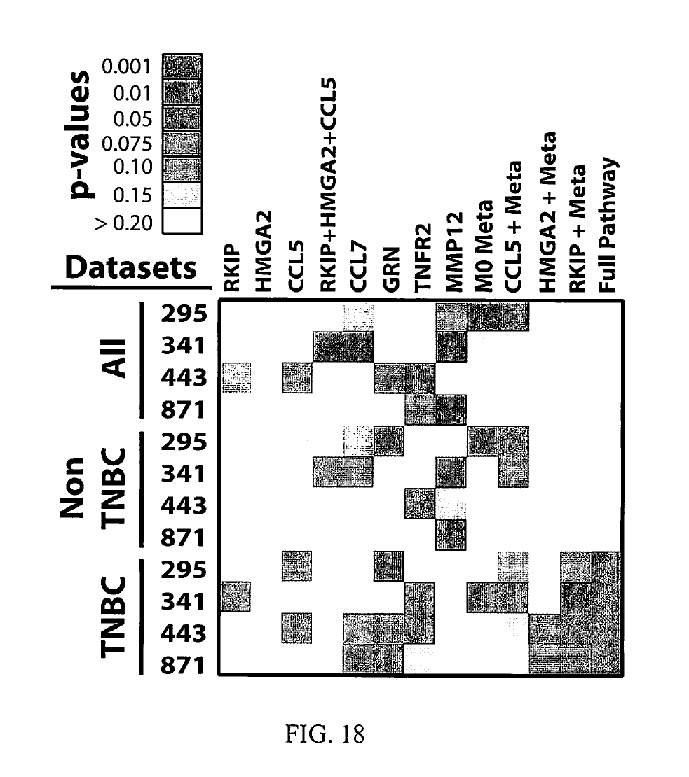

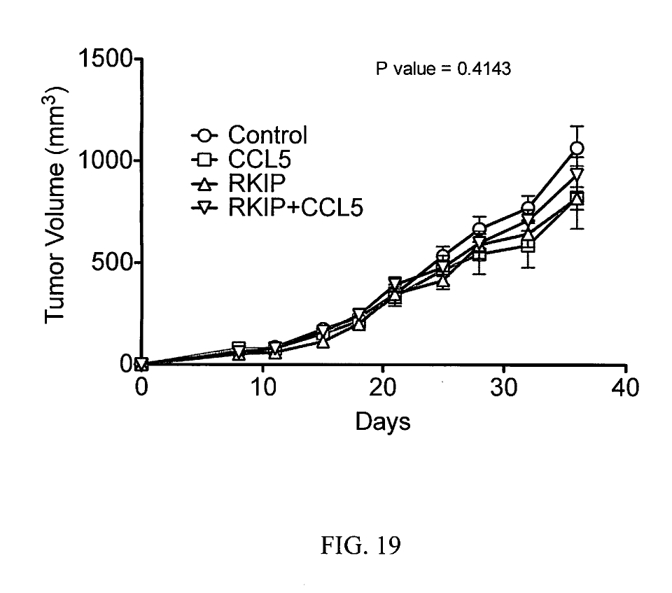

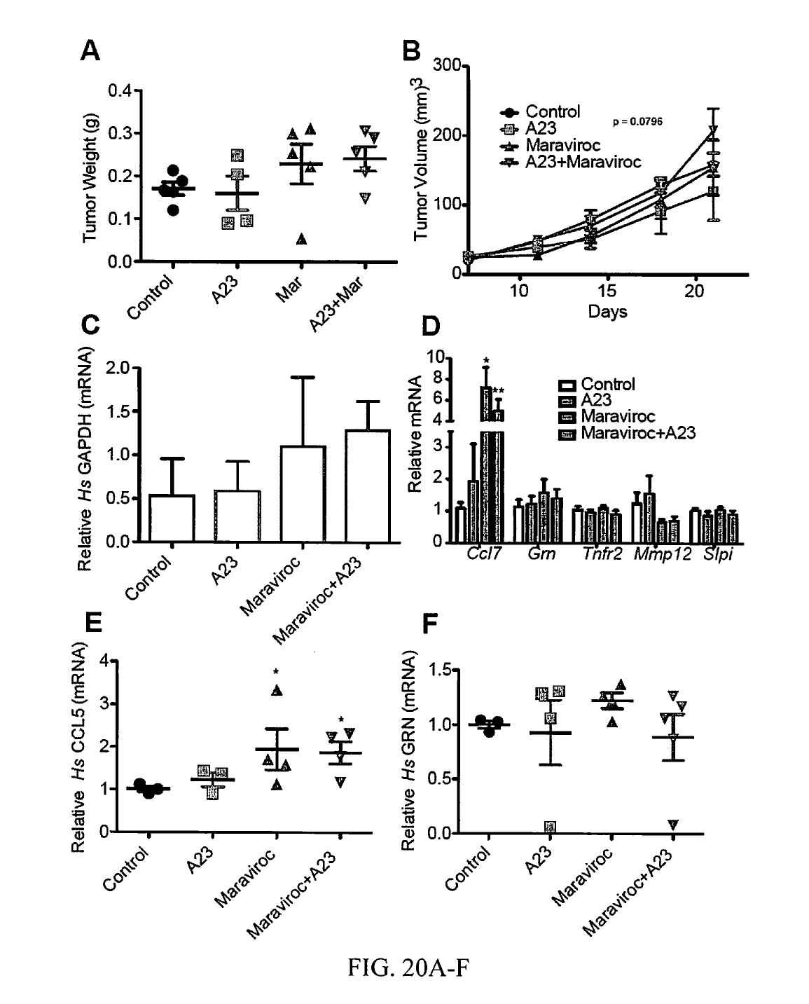

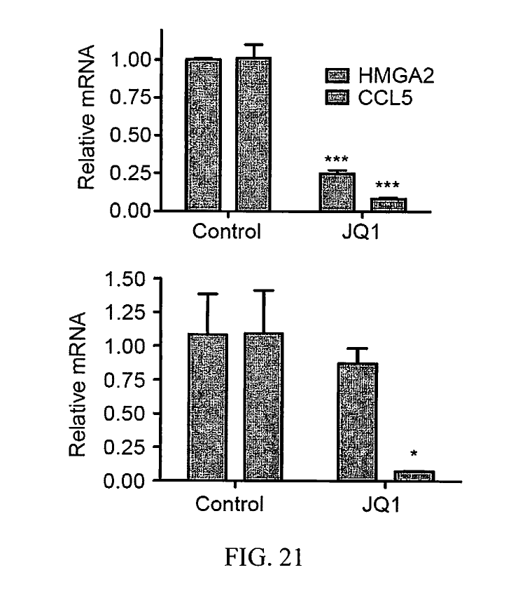

Data described herein suggests that an RKIP-HMGA2-CCL5 pathway regulates recruitment of a TAM population that promotes tumor metastasis in mice. To begin to validate this pathway in humans, Applicants analyzed gene expression in human tumors obtained from TNBC (n=319) and non-TNBC (n=1631) patients. When Applicants examined gene expression across 4 independent data sets from breast cancer patients, it was found that RKIP was induced and HMGA2, CCL5 and CCR5 were suppressed in non-TNBC tumors relative to TNBC tumors (FIG. 16C-F). Thus, an RKIP-HMGA2-CCL5 pathway can be used to classify metastatic versus non-metastatic tumors in both mouse models and human patients.

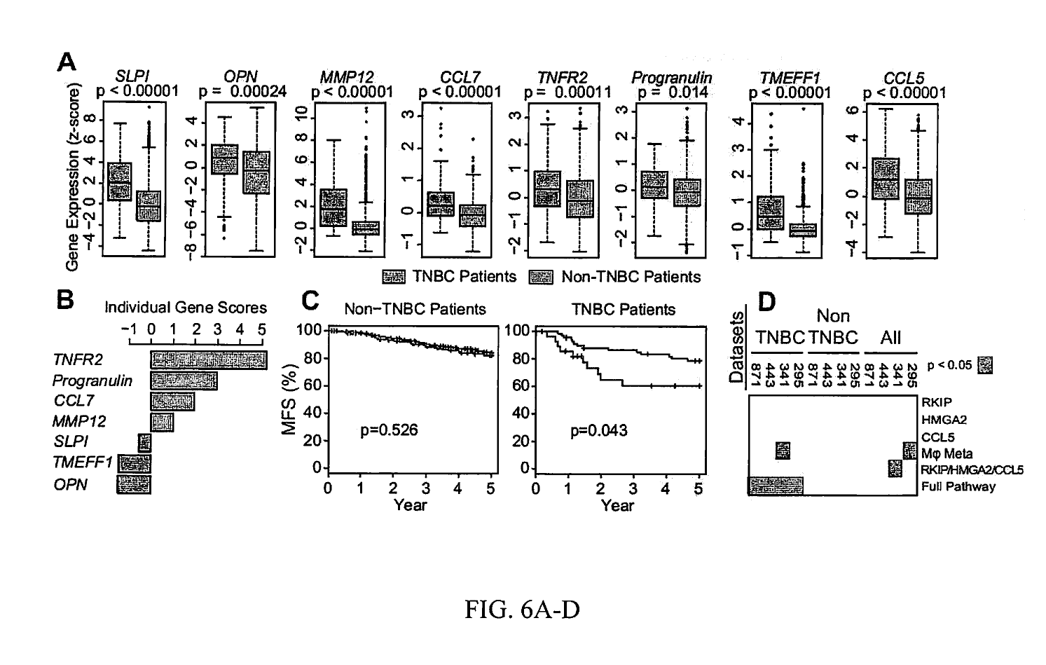

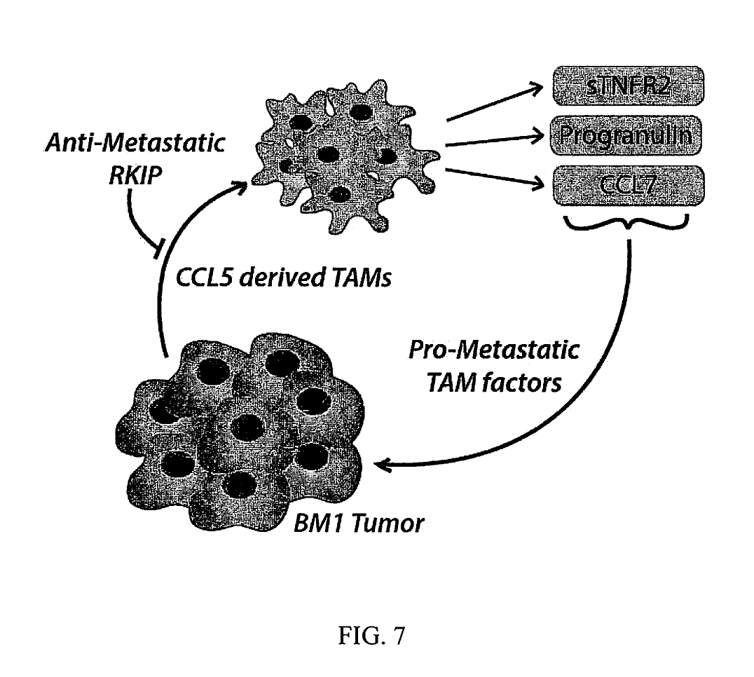

Since TAMs secrete regulators of metastasis in mice (FIG. 5), Applicants examined the expression of these TAM-secreted proteins in human breast cancer patients and found that SLPI, OPN, MMP12, CCL7, TNFR2, GRN, TMEFF1, and CCL5 were all significantly increased in TNBC compared to non-TNBC patients (FIG. 6A). Applicants performed gene set analysis (GSA) as previously described (Yun J, Frankenberger C A, Kuo W L, Boelens M C, Eves E M, Cheng N, et al. Signalling pathway for RKIP and Let-7 regulates and predicts metastatic breast cancer. EMBO J. 2011; 30:4500-14) to identify which of these factors were consistently co-expressed with CCL5 in human TNBC tumors and found a strong correlation between the expression levels of CCL5, TNFR2, GRN, and CCL7 in TNBC patients in all 4 datasets (FIG. 6B; 17). These results raise the possibility that the signaling pathway from RKIP to the three factors secreted by TAMs (TNFR2, GRN, and CCL7) defines a set of linked events that are prognostic for patient outcome.

To determine the clinical value of these genes, Applicants developed a signature utilizing the expression levels of tumor genes regulating TAM recruitment (RKIP, HMGA2, CCL5) in combination with stromal TAM-secreted genes (a TAM metagene derived from TNFR2, GRN, and CCL7). When Applicants examined all patients in the data set or those categorized as non-TNBC using molecular phenotypes as classifiers (Lehmann B D, Bauer J A, Chen X, Sanders M E, Chakravarthy A B, Shyr Y, et al. Identification of human triple-negative breast cancer subtypes and preclinical models for selection of targeted therapies. J Clin Invest. 2011; 121:2750-67), no significant relationship to clinical outcome was observed (FIG. 6D; 18). However, when Applicants limited analyses to TNBC patients, a gene signature based upon the combination of RKIP^{low} HMGA2^{high}, CCL5^{high}, and TAM-metagene^{high} expression was significantly prognostic for poor metastasis-free survival (MFS) (FIG. 6D). When considered alone, both the tumor-based gene signature (RKIP/HMGA2/CCL5) and the TAM genes (TNFR2, GRN, CCL7) were poor prognostic indicators for breast cancer outcome. Only the gene signature based on the combined tumor and TAM regulatory modules was significant across four independent sets of TNBC patients (FIG. 6C). These results highlight the importance of tumor-stromal crosstalk in the metastatic progression of TNBCs.

### B. Discussion

In this study, Applicants identified a novel mechanism whereby RKIP regulates tumor invasiveness indirectly by inhibiting infiltration of a subset of TAMs that secrete pro-metastatic factors. Applicants showed that TAMs recruited to metastatic RKIP-tumors, relative to non-metastatic RKIP+ tumors, had reduced ability to drive tumor invasion and decreased secretion of numerous pro-metastatic factors. Applicants established that RKIP inhibits TAM recruitment by reducing CCL5 expression, and further demonstrated that CCL5 overexpression was sufficient to rescue recruitment of pro-metastatic TAMs and tumor cell intravasation on a non-metastatic RKIP+ background. Interestingly, a gene signature based on the RKIP regulatory pathway, combined with pro-metastatic TAM factors was prognostic for metastasis-free survival of TNBC patients. Thus, suppression of RKIP, through direct effects in tumor cells and indirect regulation of TAM recruitment in the microenvironment, may in part explain the aggressive tumors observed in TNBC patients.

Applicant's demonstration that RKIP expression in tumors markedly attenuates infiltration of pro-metastatic TAMs suggests that metastasis suppressors play a more extensive role in regulating the tumor microenvironment than previously realized. TAMs are known to promote metastatic progression through a variety of mechanisms including recruitment of endothelial cells, secretion of ECM remodeling metalloproteinases, secretion of growth factors, suppression of the immune system by secreted factors, and co-migration along collagen fiber tracks to facilitate intravasation, extravasation and colonization (Wynn T A, Chawla A, Pollard J W. Macrophage biology in development, homeostasis and disease. Nature. 2013; 496:445-55). Applicant's protein array analysis of secreted factors suppressed in TAMs from RKIP+ tumors and restored in TAMs from RKIP+CCL5 tumors revealed similar categories including angiogenesis, extracellular matrix organization, growth factor activity, immune system development and regulation of locomotion (Table 3) (Huang da W, Sherman B T, Lempicki R A. Systematic and integrative analysis of large gene lists using DAVID bioinformatics resources. Nature protocols. 2009; 4:44-57). A positive feedback loop between tumor and stroma would lead to apparent coregulation of gene expression among different cells within a common microenvironment. Consistent with this prediction, a related study from Applicant's laboratory reveals a positive correlation between tumor and stromal gene expression not only in mouse tumor models but also in breast cancer patients. Applicant's present results show that recruitment of macrophages to the tumor microenvironment is one mechanism enabling correlated tumor and stroma gene expression.

TABLE 2
Gene lists from DAVID analysis of genes downregulated
in RKIP derived TAMs with a Benjamini p-value < 0.05

| | | | | |
|---|---|---|---|---|
| | Fold | | | |
| Term | Enrichment | Bonferroni | Benjamini | FDR |

Enrichment Score: 12.258811313832885

| | | | | |
|---|---|---|---|---|
| signal | 5.072967033 | 1.16E−12 | 1.16E−12 | 1.51E−11 |
| signal peptide | 5.040791209 | 2.19E−12 | 2.19E−12 | 1.91E−11 |
| disulfide bond | 5.327193027 | 5.28E−11 | 2.64E−11 | 4.61E−10 |
| disulfide bond | 5.168677936 | 5.93E−11 | 2.96E−11 | 7.72E−10 |
| glycoprotein | 3.818235956 | 6.27E−10 | 2.09E−10 | 8.17E−09 |
| glycosylation site:N- | 3.802364806 | 7.49E−09 | 2.50E−09 | 6.55E−08 |
| linked (GlcNAc . . .) | | | | |

Enrichment Score: 9.827688394348792

| | | | | |
|---|---|---|---|---|
| GO:0044421~extracellular | 8.383256173 | 1.32E−10 | 1.32E−10 | 2.33E−09 |
| region part | | | | |
| Secreted | 6.914382982 | 4.89E−09 | 1.22E−09 | 6.38E−08 |
| GO:0005576~extracellular | 4.474995393 | 7.90E−08 | 3.95E−08 | 1.39E−06 |
| region | | | | |
| GO:0005615~extracellular | 8.984374155 | 1.38E−07 | 4.60E−08 | 2.43E−06 |
| space | | | | |

Enrichment Score: 7.500054089287955

| | | | | |
|---|---|---|---|---|
| GO:0005615~extracellular | 8.984374155 | 1.38E−07 | 4.60E−08 | 2.43E−06 |
| space | | | | |
| GO:0005125~cytokine | 19.72725546 | 7.20E−06 | 2.40E−06 | 8.70E−05 |
| activity | | | | |
| cytokine | 26.56767956 | 1.32E−05 | 2.19E−06 | 1.71E−04 |

Enrichment Score: 5.362968602769697

| | | | | |
|---|---|---|---|---|
| GO:0030097~hemopoiesis | 16.98430634 | 1.27E−04 | 4.25E−05 | 3.27E−04 |
| GO:0048534~hemopoietic | 15.41652422 | 2.46E−04 | 6.14E−05 | 6.31E−04 |
| or lymphoid organ | | | | |
| development | | | | |
| GO:0002520~immune | 14.52281267 | 3.67E−04 | 7.35E−05 | 9.44E−04 |
| system development | | | | |
| GO:0030099~myeloid | 26.93747511 | 0.015786886 | 0.00176653 | 0.04087197 |
| cell differentiation | | | | |
| GO:0002573~myeloid | 58.94553377 | 0.020487698 | 0.001723553 | 0.053165757 |
| leukocyte differentiation | | | | |
| GO:0002521~leukocyte | 19.12355103 | 0.058964291 | 0.003370651 | 0.156008125 |
| differentiation | | | | |

Enrichment Score: 3.5055589123901614

| | | | | |
|---|---|---|---|---|
| GO:0008083~growth | 23.89325972 | 1.92E−06 | 1.92E−06 | 2.32E−05 |
| factor activity | | | | |
| growth factor | 36.70801527 | 1.92E−06 | 3.84E−07 | 2.50E−05 |
| GO:0031093~platelet | 57.73261066 | 7.25E−05 | 1.81E−05 | 0.001277903 |
| alpha granule lumen | | | | |
| GO:0060205~cytoplasmic | 53.7962963 | 9.67E−05 | 1.93E−05 | 0.001705938 |
| membrane-bounded | | | | |
| vesicle lumen | | | | |
| GO:0031983~vesicle | 51.45732689 | 1.16E−04 | 1.93E−05 | 0.002045042 |
| lumen | | | | |
| GO:0031091~platelet | 42.26851852 | 2.57E−04 | 3.68E−05 | 0.004539815 |
| alpha granule | | | | |
| mitogen | 70.45787546 | 0.001785513 | 2.55E−04 | 0.023280174 |
| GO:0005172~vascular | 360.6388889 | 0.002075263 | 5.19E−04 | 0.025107646 |
| endothelial growth factor | | | | |
| receptor binding | | | | |

TABLE 3
Gene lists from DAVID analysis of genes upregulated in RKIP + CCL5
derived (rescue) TAMs with a Benjamini p-value < 0.05

| | | | | |
|---|---|---|---|---|
| | Fold | | | |
| Term | Enrichment | Bonferroni | Benjamini | FDR |

Enrichment Score: 15.388532585594694

| | | | | |
|---|---|---|---|---|
| Secreted | 8.875227838 | 1.59E−16 | 7.97E−17 | 1.94E−15 |
| GO:0044421~extra | 10.60174419 | 1.01E−15 | 1.01E−15 | 2.16E−14 |
| cellular region part | | | | |
| GO:0005615~extra | 13.76418787 | 1.47E−14 | 7.33E−15 | 3.11E−13 |
| cellular space | | | | |
| GO:0005576~extra | 5.814732143 | 4.40E−14 | 1.47E−14 | 9.33E−13 |
| cellular region | | | | |
| cytokine | 35.60119641 | 9.49E−13 | 2.37E−13 | 1.16E−11 |
| GO:0005125~cytokine | 30.54712644 | 3.20E−12 | 3.20E−12 | 3.94E−11 |
| activity | | | | |

Enrichment Score: 6.269752254869305

| | | | | |
|---|---|---|---|---|
| GO:0006955~immune | 13.02869666 | 4.17E−09 | 4.17E−09 | 9.29E−09 |
| response | | | | |
| GO:0006954~inflammatory | 17.53290323 | 1.44E−05 | 7.19E−06 | 3.20E−05 |
| response | | | | |
| GO:0006952~defense | 9.783986175 | 2.21E−04 | 7.37E−05 | 4.92E−04 |
| response | | | | |
| GO:0009611~response | 11.36859719 | 4.03E−04 | 1.01E−04 | 8.97E−04 |
| to wounding | | | | |
| inflammatory response | 26.92911011 | 0.034827459 | 0.003538564 | 0.431429647 |
| mmu04621:NOD-like receptor | 14.2382134 | 0.087053549 | 0.015065045 | 2.101347968 |
| signaling pathway | | | | |

Enrichment Score: 4.014819061525449

| | | | | |
|---|---|---|---|---|
| GO:0042330~taxis | 24.12784848 | 0.002476462 | 2.75E−04 | 0.005519642 |
| GO:0006935~chemotaxis | 24.12784848 | 0.002476462 | 2.75E−04 | 0.005519642 |
| IPR000827:Small | 99.51260504 | 7.59E−04 | 7.59E−04 | 0.008428996 |
| chemokine, C-C group, | | | | |
| conserved site | | | | |
| PIRSF001950:small inducible | 64.57142857 | 7.87E−04 | 7.87E−04 | 0.021812987 |
| chemokine, C/CC types | | | | |
| IPR001811:Small chemokine, | 56.48012719 | 0.004338406 | 0.002171561 | 0.048246594 |
| interleukin-8-like | | | | |
| GO:0008009~chemokine | 48.2323049 | 0.00590347 | 0.001479146 | 0.072852367 |
| activity | | | | |
| GO:0042379~chemokine | 46.99557913 | 0.006384884 | 0.001280251 | 0.078810011 |
| receptor binding | | | | |
| GO:0007626~locomotory | 11.00391416 | 0.10260465 | 0.004500624 | 0.240709245 |
| behavior | | | | |
| chemotaxis | 33.87855787 | 0.017818444 | 0.001995684 | 0.219050316 |
| GO:0007610~behavior | 7.575945838 | 0.136952694 | 0.00587411 | 0.327342147 |
| SM00199:SCY | 31.84306888 | 0.00712501 | 0.00712501 | 0.197961854 |
| inflammatory response | 26.92911011 | 0.034827459 | 0.003538564 | 0.431429647 |
| mmu04621:NOD-like receptor | 14.2382134 | 0.087053549 | 0.015065045 | 2.101347968 |
| signaling pathway | | | | |
| mmu04062:Chemokine | 6.062975486 | 0.25000158 | 0.028358547 | 6.488114962 |
| signaling pathway | | | | |

Enrichment Score: 3.400529437607057

| | | | | |
|---|---|---|---|---|
| GO:0031401~positive | 33.71712159 | 4.70E−04 | 9.40E−05 | 0.00104612 |
| regulation of protein | | | | |
| modification process | | | | |
| GO:0050731~positive | 62.61751152 | 6.49E−04 | 1.08E−04 | 0.001446074 |
| regulation of | | | | |
| peptidyl-tyrosine | | | | |
| phosphorylation | | | | |
| lymphokine | 175.0392157 | 1.11E−04 | 1.39E−05 | 0.001358392 |
| GO:0032270~positive | 26.29935484 | 0.001618838 | 2.02E−04 | 0.003606619 |
| regulation of cellular | | | | |
| protein metabolic process | | | | |
| mmu04630:Jak-STAT | 11.61538462 | 1.00E−04 | 5.01E−05 | 0.00233471 |
| signaling pathway | | | | |
| GO:0051247~positive | 24.12784848 | 0.002476462 | 2.75E−04 | 0.005519642 |
| regulation of protein | | | | |
| metabolic process | | | | |
| GO:0050730~regulation | 41.35118685 | 0.003527811 | 3.53E−04 | 0.007866984 |
| of peptidyl-tyrosine | | | | |
| phosphorylation | | | | |
| GO:0001934~positive | 39.1359447 | 0.004402706 | 4.01E−04 | 0.009822207 |
| regulation of protein | | | | |
| amino acid phosphorylation | | | | |
| GO:0042327~positive | 36.52688172 | 0.005805485 | 4.85E−04 | 0.012960655 |
| regulation of phosphorylation | | | | |
| GO:0045937~positive | 35.34859521 | 0.006618343 | 5.11E−04 | 0.014781251 |
| regulation of phosphate | | | | |
| metabolic process | | | | |
| GO:0010562~positive | 35.34859521 | 0.006618343 | 5.11E−04 | 0.014781251 |
| regulation of phosphorus | | | | |
| metabolic process | | | | |
| GO:0031399~regulation | 15.93900293 | 0.018494241 | 0.00103654 | 0.041547649 |
| of protein modification | | | | |
| process | | | | |

On a molecular level, one mechanism by which the metastasis suppressor RKIP regulates TAM recruitment is by attenuating CCL5 expression. Although the CCL5-CCR5 axis has been implicated in breast cancer metastasis, the role of macrophages in this process and the molecular and cellular mechanisms of action have been controversial (Karnoub A E, Dash A B, Vo A P, Sullivan A, Brooks M W, Bell G W, et al. Mesenchymal stem cells within tumour stroma promote breast cancer metastasis. Nature. 2007; 449:557-63 and Velasco-Velazquez M, Jiao X, De La Fuente M, Pestell T G, Ertel A, Lisanti M P, et al. CCR5 antagonist blocks metastasis of basal breast cancer cells. Cancer Res. 2012; 72:3839-50). Applicant's study shows that CCL5 acts in a paracrine fashion to recruit a subset of TAMs that have a pro-metastatic function, but are phenotypically distinct from the classic M1 and M2 macrophages. Expression of CCL5 and the presence of CCL5-recruited TAMs are insufficient by themselves to predict outcome, but the combination of the RKIP tumor signaling pathway with the CCL5-TAMs enable generation of a prognostic gene signature for TNBC patients. This result suggests that taking into account both tumor and stromal factors may be effective for prognosis and therapeutic efficacy in TNBC patients.

Consistent with this hypothesis, previous work has shown that shed TNFR2 (sTNFR2) protein is higher in the plasma of pancreatic, endometrial, and breast cancer patients (Grote V A, Kaaks R, Nieters A, Tjonneland A, Halkjaer J, Overvad K, et al. Inflammation marker and risk of pancreatic cancer: a nested case-control study within the EPIC cohort. Br J Cancer. 2012; 106:1866-74; Dossus L, Becker S, Rinaldi S, Lukanova A, Tjonneland A, Olsen A, et al. Tumor necrosis factor (TNF)-alpha, soluble TNF receptors and endometrial cancer risk: the EPIC study. Int J Cancer. 2011; 129:2032-7; and Gross A L, Newschaffer C J, Hoffman-Bolton J, Rifai N, Visvanathan K. Adipocytokines, inflammation, and breast cancer risk in postmenopausal women: a prospective study. Cancer Epidemiol Biomarkers Prev. 2013; 22:1319-24) and is associated with an increased risk of cancer (Gross A L, Newschaffer C J, Hoffman-Bolton J, Rifai N, Visvanathan K. Adipocytokines, inflammation, and breast cancer risk in postmenopausal women: a prospective study. Cancer Epidemiol Biomarkers Prev. 2013; 22:1319-24). Progranulin (GRN) expression blocks TNFR2-mediated inflammation and has been shown to drive migration, invasion and VEGF expression in breast cancer (He Z, Ismail A, Kriazhev L, Sadvakassova G, Bateman A. Progranulin (PC-cell-derived growth factor/acrogranin) regulates invasion and cell survival. Cancer Res. 2002; 62:5590-6 and Tangkeangsirisin W, Serrero G. PC cell-derived growth factor (PCDGF/GP88, progranulin) stimulates migration, invasiveness and VEGF expression in breast cancer cells. Carcinogenesis. 2004; 25:1587-92). Moreover, GRN is highly expressed in a number of tumors including breast, and has also been targeted using biologics in hepatocellular carcinoma (He Z, Bateman A. Progranulin (granulin-epithelin precursor, PC-cell-derived growth factor, acrogranin mediates tissue repair and tumorigenesis. Journal of molecular medicine. 2003; 81:600-12 and Ho J C, Ip Y C, Cheung S T, Lee Y T, Chan K F, Wong S Y, et al. Granulin-epithelin precursor as a therapeutic target for hepatocellular carcinoma. Hepatology. 2008; 47:1524-32). Because of the strong evidence of pro-invasive action and the clinical relevance of these factors, CCL5, GRN, sTNFR2, and CCL7 are all potential targets for anti-TNBC drug treatment.

While Applicant's studies revealed a role for RKIP in macrophage recruitment to tumors in both xenograft and syngeneic mouse models, other metastasis suppressors might have unique roles in regulating additional cell types in the stroma. Furthermore, Applicants identified factors secreted by pro-metastatic TAMs in this study using immune compromised nude mice that lack mature Tcells. It is therefore possible that, in TNBC patients, RKIP might also play a role in regulating Tcells, through factors such as CD80, which is upregulated in RKIP+ BM1-derived TAMs and is a potential therapeutic tool in the treatment of breast cancer patients (Dols A, Smith J W, 2nd, Meijer S L, Fox B A, Hu H M, Walker E, et al. Vaccination of women with metastatic breast cancer, using a costimulatory gene (CD80)-modified, HLA-A2-matched, allogeneic, breast cancer cell line: clinical and immunological results. Human gene therapy. 2003; 14:1117-23).

Understanding how the tumor cells and TAMs interact could lead to novel strategies for blocking TAM recruitment to TNBCs and tumor metastatic progression. In the case of CCL5-recruited TAMs, there is an array of pro-metastatic genes that support the tumor through several different pathways. Elegant work on TAMs in breast cancer by Pollard and others has implicated the role of CCL2 and GM-C SF in TAM recruitment in breast cancer. However, the models used to study CCL2 are based on the luminal/HER2+ MMTV-PyVT GEM model (Qian B Z, Li J, Zhang H, Kitamura T, Zhang J, Campion L R, et al. CCL2 recruits inflammatory monocytes to facilitate breast-tumour metastasis. Nature. 2011; 475:222-5) and are likely to display a unique set of molecular interactions. In the present study, no inhibition of CCL2 or GMCSF expression by RKIP was observed by RNAseq analysis. Instead, Applicants show that expression of a metastasis suppressor in tumor cells regulates recruitment of a specific TAM population secreting pro-metastatic factors. Future studies will be necessary to determine which inhibitors of these pathways will be most effective therapeutically either alone or in combination in triplenegative breast cancer.

### C. Methods

1. Cell Culture

BM1, MDA-MD-436, and 4T1.2 cell lines were cultured in DMEM media supplemented with 10% fetal bovine serum, 50 U/ml penicillin, and 50 μg/ml streptomycin. Cells were transduced with lentiviral vectors for shRNA knockdown or overexpression from GE/Dharmacon. Cells were selected for 14 days using 3 μg/ml of puromycin or 10 μg/ml of blasticidin after lentiviral transduction before use.

2. Invasion Assays

2×105 BM1 cells were plated in 24-well BD trans-well inserts coated with growth factor depleted matrigel as previously described (Dangi-Garimella S, Yun J, Eves E M, Newman M, Erkeland S J, Hammond S M, et al. Raf kinase inhibitory protein suppresses a metastasis signalling cascade involving LIN28 and let-7. EMBO J. 2009; 28:347-58). After 24 hours inserts were transferred to a new well and stained with 4 ng/μ1 of Calcein AM for one hour. Stained cells were then dissociated using gentle shaking for one hour at 37° C. and 150 RMP in dissociation buffer from Trevigen. Fluorescence was measured using a Victor X3 fluorescent plate reader with excitation at 465 nm and emission at 535 nm.

3. Tumor Associated Macrophage Isolations

Tumors were grown to approximately 0.2 g before being harvested. Tumors were physically dissociated, then chemically dissociated using a combination of collagenase, hyaluronidase, and DNAse while shaking at 150 RPM for 2 hours. Cells were then filtered through a 70 μm mesh filter. Mononuclear cells were isolated using Ficoll-Paque PREMIUM (GE Healthcare) gradient centrifugation at 420 RPM for 40 minutes. Macrophages were then obtained using CD11b positive selection beads from Milteny Biotech.

4. Conditioned Media

For THP-1 conditioned media, 5×10⁶ THP-1 cells were plated in a T-75 flask with 5 mL of 10% serum containing DMEM. Media was collected after 24 hours and cells and cell debris were removed by centrifugation.

For tumor derived macrophages, 5×10⁵ TAMs were plated in one well of a 6-well plate. After 30 minutes, cells were washed with PBS to ensure only viable macrophages attached to the plate remained. Cells were incubated for 24 hours to obtain conditioned media in serum free DMEM. Cells and cell debris were removed by centrifugation.

5. Mice

Balb/c nude mice were purchased from Harlan. Mice were injected with 1×10⁶ human or 5×10⁵ mouse tumor cells. Tumor volumes were measured twice per week and calculated as volume=(π/6)×width×length. Tumors were grown to ˜200 cm³ and removed for analysis.

C57BL/6J-Wnt1-Hmga2^{-/-} and C57BL/6J-Wnt1-Hmga2^{+/+} mice have been described previously. Briefly, C57BL/6J-backcrossed Hmga2^{+/-} female mice were mated with C57BL/6J-Wnt1 male mice (The Jackson Laboratory). Wnt1 transgenic, Hmga2^{+/-} male mice were then mated with Wnt transgenic, Hmga2^{+/-} female littermates to obtain transgenic mice in the Hmga2^{+/+}, Hmga2^{+/-}, and Hmga2^{-/-} genetic backgrounds. PCR-based genotyping was performed for the Hmga2 and Wnt locus.

6. Categorical Analysis of RNAseq mRNA Expression

To perform ontological enrichment analyses we compared the specified set of significant genes to the total set of successfully-modeled genes in the corresponding dataset. Applicants identified enriched categories with the GOseq function implemented in GOseq using the default settings (Wallenius approximation), and corrected p-values for multiple testing using the Benjamini and Hochberg approach.

Applicants used GOseq to determine the ontological categories enriched within sets of genes whose boundaries may be clearly defined (e.g., differential expression at an FDR of 0.01). For statistics that are continuous and in which appropriate cutoff values are less clear, such as correlation coefficients, Applicants used GSEA with the statistics considered as a ranked list using the default settings.

7. Enrichment of Differential Expression in Cell Type-Specific Gene Sets

To determine whether differences in gene expression observed in Applicant's data were consistent with infiltration of specific cell types into the tumor microenvironment, Applicants used the RefDIC database of mouse immune cell expression data (Hijikata A, Kitamura H, Kimura Y, Yokoyama R, Aiba Y, Bao Y, et al. Construction of an open-access database that integrates cross-reference information from the transcriptome and proteome of immune cells. Bioinformatics. 2007; 23:2934-41) to identify genes whose expression was highly specific to a particular immune cell type, and then determined whether there was disproportionate evidence that these genes were differentially expressed within the set of Pvalues quantifying the evidence for altered expression levels between metastatic and nonmetastatic stroma tissue. To do so, Applicants used the RefDIC Specific Gene Finder tool using the default settings to extract the set of genes whose expression is specifically upregulated in each of 13 specific immune cell types. Each cell type was analyzed using all P-values from genes specific to the cell type in question, with all of the P-values used as the background set. The details of the specific cell types queried and the number of specific genes identified are included in Table 3.

Applicants determined whether each set of genes specifically upregulated within each cell type was disproportionately differentially expressed within our data using an approach combining bootstrapping with standard visualization using a quantile-quantile plot (Q-Q plot). In a standard Q-Q plot an observed distribution of n values (typically P-values) is compared to the theoretical distribution by ordering the observed values and plotting them against n ordered random draws from the theoretical distribution as the respective independent and dependent variables in a 2d plot. In this visualization strategy, deviations above the line x=y correspond to an enrichment of values more extreme than are expected to result from random draws from the theoretical distribution.

Applicants used a bootstrapping strategy to compare the P-values observed for the genes specific to each immune cell type to the overall distribution of P-values summarizing the evidence for differential expression between metastatic and non-metastatic stroma. For each gene set containing n genes whose expression is specific to a particular cell type, Applicants randomly sampled 100 sets of n P-values from the full distribution using the weights predicted by the PWF generated above, rank-ordered them, and then took the mean and standard deviation of the Pvalues at each rank. Applicants then used these values as the dependent variables in the Q-Q plot, and determined divergence from x=y in excess of the standard deviation by inspection.

8. Immunohistochemistry

Tissue sections were deparaffinized and rehydrated through xylenes and serial dilutions of EtOH to distilled water, and then incubated in antigen retrieval buffer at 97° C. for 20 minutes. Primary and secondary antibody incubations were carried out in a humidity chamber at room temperature, and detected using an Elite kit (PK-6100, Vector Laboratories) and DAB (DAKO, K3468) system according to the manufacturers' protocols. Following staining, tissue sections were briefly immersed in hematoxylin for counterstaining and were covered with cover glasses. Stained tissue sections were scanned at 20× magnification and analyzed using Aperio Imagescope ePathology® software. To quantify infiltrating macrophages, the number of pixels positive for staining were normalized to total number of pixels inside the tumor stroma border.

9. Patient Data Classification

Patients were classified into TNBC and non-TNBC categories using an algorithm previously described. Briefly, mRNA expression of estrogen, progesterone and her2/neu receptor genes was modeled as bimodal distribution of two Gaussian peaks then binned into positive or negative expressing patients. Those patients classified as absent all three receptors were categorized triple negative.

10. Patient Data Correlation

mRNA expression levels of genes were compared between TNBC and non-TNBC patients using a Student's T-Test to predict if a significant difference exists in the in the mean gene expression of the two patient populations.

11. Patient Data Survival

Applicants combined RKIP, HMGA2, CCL5 genes with a macrophage metagene to produce a simple classifier representing activation of a signaling pathway defined by the experimental results represented in this manuscript. The four components were designated independently as high or low as to whether they are above (in the case of HMGA2, CCL5 and the macrophage metagene) or below (in the case of RKIP) median expression across each data set for the level of mRNA in each component in each patient. For patients that have 3 of the 4 components designated, Applicants declare the pathway to be “active” and stratify in to two groups based on this. Applicants tested the significance of the association to metastasis free survival in the framework of the model using a log rank test.

### Example 2: Therapeutic Targeting of Ccl5 and Progranulin

Maraviroc resistance figure: targeting CCL5/CCR5 through Maraviroc alone is not effective. Tumor cells secreted more CCL5 in response to therapy and that we then saw more CCL7 in the tumor-associated macrophages. The Progranulin blocking antibody A23 was not effective in the current experiment. However, it is likely that it may only bind and block human progranulin, but not mouse progranulin. Therefore, it is contemplated that treatment with an agent that reduces the expression or activity of progranulin and an agent that reduces the expression or activity of CCL5 can act synergistically to treat breast cancer.

To overcome resistance to Maraviroc and increased CCL5 levels, the inventors are treating the tumor cells with JQ1, a bromodomain inhibitor which regulates HMGA2. HMGA2 is an upstream regulator of CCL5. Additionally, both genes expression levels are included in our prognostic signature. The attached figure shows cells treated with 300 nM JQ1 in both human TNBC BM1 cells as well as mouse basal-like M6C cells show a significant reduction of CCL5 expression. We plan to in the future test whether JQ1 in conjunction with Maraviroc are able to lead to a reduction in metastasis in a mouse model.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating metastatic breast cancer in a patient in need thereof, the method comprising:
administering a treatment that inhibits or reduces the expression level or protein activity of one or more of RKIP, HMGA2, CCL5, TNFR2, GRN, and CCL7.

2. The method of claim 1, wherein the treatment inhibits or reduces the expression level or protein activity of CCL5, GRN, or both of CCL5 and GRN.

3. The method of claim 2, wherein the treatment inhibits or reduces the expression level or protein activity of CCL5.

4. The method of claim 2, wherein the treatment inhibits or reduces the expression level or protein activity of GRN.

5. The method of claim 2, wherein the treatment inhibits or reduces the expression level or protein activity of both CCL5 and GRN.

6. The method of claim 3 wherein the treatment comprises maraviroc.

7. The method of claim 3, wherein the treatment comprises an EGFR inhibitor.

8. The method of claim 7, wherein the EGFR inhibitor is tarceva.

9. The method of any one of claims 2-8, wherein the treatment further comprises a second therapeutic agent.

10. The method of claim 9, wherein the second therapeutic agent is a bromodomain inhibitor.

11. The method of claim 10, wherein the bromodomain comprises a protein listed in Table 1.

12. The method of claim 10, wherein the inhibitor comprises JQ1, I-BET 151 (GSK1210151A), I-BET 762 (GSK525762), OTX-015, TEN-010 (Tensha therapeutics), CPI-203, CPI-0610, RVX-208 (Resverlogix Corp), LY294002, or combinations thereof

13. The method of claim 10, wherein the inhibitor is a small molecule inhibitor, a polypeptide inhibitor, an antagonistic antibody, or a nucleic acid inhibitor.

14. The method of claim 4, wherein the treatment is an anti-GRN antibody.

15. The method of claim 4, wherein the treatment comprises a small molecule inhibitor, a nucleic acid inhibitor, or a polypeptide inhibitor.

16. The method of any one of claim 1-8 or 10-15, wherein the subject is diagnosed with or determined to have metastatic breast cancer.

17. The method of claim 9, wherein the subject is diagnosed with or determined to have metastatic breast cancer.

18. The method of claim 16, wherein the subject has or was determined to have a reduced expression level of RKIP and/or an elevated expression level of one or more of HMGA2, CCL5, and TAM-metagene.

19. The method of claim 17, wherein the subject has or was determined to have a reduced expression level of RKIP and/or an elevated expression level of one or more of HMGA2, CCL5, and TAM-metagene.

20. The method of claim 18 or 19, wherein the expression level is elevated or reduced relative to a control level of expression.

21. The method of claim 20, wherein the control level of expression is the level of expression in non-metastatic breast cancer.

22. The method of claim 20, wherein the control level of expression is the level of expression in non-cancerous tissue.

23. The method of any one of claims 1-22, wherein the subject is or was determined to have an elevated expression level of CCL5.

24. The method of any one of claims 1-23 wherein the subject is or was determined to have an elevated expression level of GRN.

25. The method of claim 1, wherein the treatment inhibits or reduces the expression level or protein activity of CCL7.

26. The method of any one of claims 1-25, wherein the patient is determined to have a increased expression level or protein activity of one or more of RKIP, HMGA2, CCL5, TNFR2, GRN, and CCL7.

27. The method of any one of claims 1-26, wherein the method further comprises measuring the expression level or protein activity of one or more of RKIP, HMGA2, CCL5, TNFR2, GRN, and CCL7.

28. The method of any one of claims 1-27, wherein the method further comprises comparing the expression level or protein activity of one or more of RKIP, HMGA2, CCL5, TNFR2, GRN, and CCL7 in a sample from the subject to the expression level or protein activity of one or more of RKIP, HMGA2, CCL5, TNFR2, GRN, and CCL7 in a control sample.

29. A method for treating a patient determined to be at high risk for developing or having metastatic breast cancer comprising
administering adjuvant or neoadjuvant therapy to the patient determined to be at high risk for developing or having metastatic breast cancer,
wherein the patient was determined to be at high risk for developing or having metastatic breast cancer by determining that the expression level of RKIP was reduced and/or the expression level of one or more of HMGA2, CCL5, and TAM-metagene was elevated in a biological sample from the patient compared to a control non-metastatic tissue sample.

30. The method of claim 29, wherein the adjuvant or neoadjuvant therapy comprises one or more of chemotherapy, hormonal therapy, surgical removal of the breast and/or ovaries, trastuzumab, and radiation therapy.

31. The method of claim 30, wherein the chemotherapy comprises one or more of docetaxel, paclitaxel, cisplatin, carboplatin, vinorelbine, capecitabine, liposomal doxorubicin, gemcitabine, mitoxantrone, ixabepilone, albumin-bound paclitaxel, trastuzumab, tamoxifen, aromatase inhibitor, toremifene, magestrol acetate, fluvestran, and eribulin.

32. The method of claim 31, wherein the aromatase inhibitor comprises one or more of letrozole, anastrozole, and exemstane.

33. The method of any one of claims 29-32, wherein an adjuvant therapy is administered to the patient determined to be at high risk for developing or having metastatic breast cancer.

34. The method of any one of claims 29-33, wherein the patient was determined to be at high risk for developing or having metastatic breast cancer when at least three of the following are determined: reduced RKIP expression, elevated HMGA2 expression, elevated CCL5 expression, and elevated TAM-metagene expression.

35. A method for treating a patient for metastatic or non-metastatic breast cancer comprising:
treating the patient for metastatic breast cancer after the patient is determined to have an elevated expression level of one or more of HMGA2, CCL5, and TAM-metagene and/or a reduced expression level of RKIP in a biological sample from the patient relative to the expression level of the same genes in a control non-metastatic tissue sample; and
treating the patient for non-metastatic breast cancer after the patient is determined to have a reduced or substantially the same expression level of one or more of HMGA2, CCL5, and TAM-metagene and/or an elevated or substantially the same expression level of RKIP in a biological sample from the patient relative to the expression level of the same genes in a control non-metastatic tissue sample.

36. The method of claim 35, wherein the treatment for metastatic breast cancer comprises one or more of chemotherapy, hormonal therapy, surgical removal of the breast and/or ovaries, trastuzumab, and radiation therapy.

37. The method of claim 35 or 36, wherein the treatment for the patient with non-metastatic breast cancer excludes one or more of chemotherapy, surgical removal of the breast and/or ovaries, and radiation therapy.

38. The method of claim 36 or 37, wherein the chemotherapy comprises one or more of docetaxel, paclitaxel, cisplatin, carboplatin, vinorelbine, capecitabine, liposomal doxorubicin, gemcitabine, mitoxantrone, ixabepilone, albumin-bound paclitaxel, trastuzumab, tamoxifen, aromatase inhibitor, toremifene, magestrol acetate, fluvestran, and eribulin.

39. The method of claim 38, wherein the aromatase inhibitor comprises one or more of letrozole, anastrozole, and exemstane.

40. The method of any one of claims 35-39, wherein the treatment for the patient with metastatic breast cancer comprises adjuvant therapy.

41. The method of any one of claims 35-40, wherein the treatment for a patient with non-metastatic breast cancer comprises one or more of surgical removal of the primary tumor, surgical removal of the breast, radiation therapy, and neoadjuvant therapy.

42. The method of any one of claims 35-41, wherein the treatment for the patient with non-metastatic breast cancer excludes adjuvant therapy.

43. The method of any one of claims 29-42, wherein the method further comprises measuring the expression level of one or more of RKIP, HMGA2, CCL5, and TAM-metagene in a biological sample from the patient.

44. The method of any one of claims 29-43, wherein the method further comprises comparing the expression level of one or more of RKIP, HMGA2, CCL5, and TAM-metagene in a biological sample from the patient to the expression level of the same gene in a control non-metastatic tissue sample.

45. The method of any one of claims 29-44, wherein the patient is treated for metastatic breast cancer after the patient is determined to have at least three of: an elevated expression level of HMGA2, an elevated expression level of CCL5, an elevated expression level of TAM-metagene and a reduced expression level of RKIP in a biological sample from the patient relative to the expression level of the same genes in a control non-metastatic tissue sample.

46. A method for predicting a patient's prognosis for survival and/or metastasis-free survival of breast cancer comprising:
measuring the expression level of one or more of RKIP, HMGA2, CCL5, and TAM-metagene in a biological sample from the patient;
comparing the expression level of the one or more of RKIP, HMGA2, CCL5, TNFR2, GRN, and CCL7 in the biological sample from the patient to the expression level of the same gene or genes in a control non-metastatic tissue sample;
predicting that the patient is likely to survive and or have metastatic-free survival when the measured level of one or more of HMGA2, CCL5, and TAM-metagene is reduced or not substantially different and/or the measured level of RKIP is elevated or not substantially different in a biological sample from the patient relative to the expression level of the same genes in a control non-metastatic tissue sample; and
predicting that the patient is not likely to survive or have metastatic-free survival when the measured level of one or more of HMGA2, CCL5, and TAM-metagene is elevated and/or the measured level of RKIP is reduced in a biological sample from the patient relative to the expression level of the same genes in a control non-metastatic tissue sample.

47. The method of claim 46, wherein the patient is predicted to not likely survive or have metastatic-free survival after the patient is determined to have at least three of: an elevated expression level of HMGA2, an elevated expression level of CCL5, an elevated expression level of TAM-metagene and a reduced expression level of RKIP in a biological sample from the patient relative to the expression level of the same genes in a control non-metastatic tissue sample.

48. The method of any of claims 29-46, wherein the patient is determined to have a triple negative (ER⁻/PR⁻/HER2⁻) breast cancer (TNBC) subtype.

49. The method of any of claims 29-48, further comprising determining whether the breast cancer sample has a triple negative breast cancer (TNBC) subtype.

50. The method of any one of claims 29-49, wherein the biological sample from the patient is cancerous.

51. The method of any one of claims 29-50, wherein the biological sample from the patient comprises breast cancer tumor cells.

52. The method of any one of claims 29-51, wherein the control non-metastatic tissue sample comprises non-cancerous cells from the breast.

53. The method of any one of claims 29-51, wherein the control non-metastatic tissue sample comprises a reference level of expression from breast cancer tumors that are non-metastatic.

54. The method of any of claims 29-53, further comprising assaying nucleic acids in the breast cancer sample.

55. The method of claim 53, wherein assaying nucleic acids comprises using PCR, microarray analysis, next generation RNA sequencing, or a combination thereof.

56. The method of any of claims 29-55, further comprising testing protein expression in the breast sample.

57. The method of claim 56, wherein testing protein expression comprises performing ELISA, RIA, FACS, dot blot, Western Blot, immunohistochemistry, antibody-based radioimaging, mass spectroscopy, or a combination thereof.

58. The method of any of claims 29-57, further comprising recording the expression level or the prognosis score in a tangible medium.

59. The method of any of claims 29-58, further comprising reporting the expression level or the prognosis score to the patient, a health care payer, a physician, an insurance agent, or an electronic system.

60. The method of any one of claims 29-59, further comprising monitoring the patient for breast cancer recurrence or metastasis or prescribing a treatment that excludes the previously prescribed treatment.
